SEQUENCE LISTING (6 sequences)

SEQ ID NO: 1 - protein, 53 aa, Canine PLB
MDKVQYLTRSAIRRAISTIEMPQQARQNLQNLFINFCLILICLLLICIIVMLL

SEQ ID NO: 2 - protein, 53 aa, Unknown
  WSPLB
MDKVQYLTRSAIRRAISTIEMPQQARQNLQNLYINRCLREICQELKEIRAMLK

SEQ ID NO: 3 - protein, 53 aa, Unknown
  SIMM-Full
MDKVQYLTRSAIRRAISTIEMPNQARQKLQNEFINYCLKEICELLKEIKQMLK

SEQ ID NO: 4 - protein, 53 aa, Unknown
  ADA-Full
MDKVQYLTRSAIRRAISTIEMPNQARQKLQNEFINYCLKLISELLECIKQMLK

SEQ ID NO: 5 - protein, 27 aa, Unknown
  PLB-Comp1
QKLKNQEINQCLQLICELLRQIIRMLT

SEQ ID NO: 6 - protein, 27 aa, Unknown
  PLB-Conp2
QKLQNQEINQCLQLICELLRQIIRMLT

(19) United States
(12) Patent Application Publication
(10) Pub. No.: US 2004/0215400 A1
(43) Pub. Date: Oct. 28, 2004

(54) Computational design of a water-soluble analog of a protein, such as phospholamban and potassium channel KcsA

(75) Inventors: Avram M. Slovic, Philadelphia, PA (US); Christopher M. Summa, Dunmore, PA (US); Jeffery G. Saven, Philadelphia, PA (US); William F. DeGrado, Moylan, PA (US); Hidetoshi Kono, Kyoto (JP)

Correspondence Address: STERNE, KESSLER, GOLDSTEIN & FOX PLLC, 1100 NEW YORK AVENUE, N.W., WASHINGTON, DC, 20005, US

(73) Assignee: The Trustees of the University of Pennsylvania

(21) Appl. No.: 10/760,721

(22) Filed: Jan. 21, 2004

Related U.S. Application Data
(60) Provisional application No. 60/441,103, filed on Jan. 21, 2003.

(51) Int. Cl. G06F 19/00; G01N 33/48; G01N 33/50

(52) U.S. Cl. 702/19

(57) ABSTRACT

Membrane proteins and water-soluble proteins share a similar core. This similarity suggests that it should be possible to water-solubilize membrane proteins by mutating only their lipid-exposed residues. Computational tools and methods are disclosed herein that can be used to design water-soluble variants of helical membrane proteins, using the pentameric phospholamban (PLB) and potassium channel KcsA as models. To water-solublize PLB, the membrane-exposed positions were changed to polar or charged amino acids, while the putative core was left unaltered. We generated water-soluble phospholamban (WSPLB), and compared its properties to its predecessor PLB. As a probe of the correctness of the fold of the water soluble KcsA, the computationally designed proteins contain an agitoxin-2 binding site from a mammalian homologue of the channel. The resulting proteins express in high yield in E. coli and share the intended functional and structural properties with KcsA, including secondary structure, tetrameric quaternary structure, and tight, specific binding to both agitoxin2 and a small molecule channel blocker.

### STATEMENT REGARDING FEDERALLY-SPONSORED RESEARCH AND DEVELOPMENT

Part of the work performed during development of this invention utilized U.S. Government funds. The U.S. Government has certain rights in this invention. This work was supported by National Institutes of Health grants GM-60610 and GM-61267 and National Science Foundation grant CHE-99-84752.

### BACKGROUND OF THE INVENTION

FIELD OF THE INVENTION

The invention is directed to computational design of water-soluble analogs of proteins, such as phospholamban and potassium channel KcsA.

### SUMMARY OF THE INVENTION

The present invention is directed to methods, systems, and computer program products for computational design of water-soluble analogs of proteins, such as phospholamban and potassium channel KcsA. In accordance with the invention, a computational process for the water-solubilization of membrane proteins involves 1) defining or predicting the backbone structure of a membrane protein; 2) defining the residues that are in contact with the apolar regions of the phospholipid membrane; and 3) using computational methods to define a set of mutations that will confer water solubility on the structure while retaining its uniquely folded structure. These steps are described below.

1) Backbone structure. The backbone structures can be defined by experimental structure determination (e.g., X-ray crystallography, NMR, electron microscopy or diffraction) of the desired structure or of a homologous protein. Methods for modeling the structure of interest, beginning with a homologous three-dimensional structure, are well known to practitioners skilled in the art. Alternatively, the backbone structure can be or are defined by de novo structure prediction, which is often guided by experimental biological data, rates of mutation of sidechains, or site directed mutagenesis.

2) The residues contacting the apolar regions of the bilayer can be defined by computing their accessibility to a spherical probe. Often a probe of approximately 1.4 Å radius (approximating the radius of water) is used in these calculations. However, one can use larger or smaller radii, which should contact fewer or more atoms. Once the solvent-accessibility surface has been defined, it is useful to consider a threshold above which a residue is considered to be exposed. This threshold can be expressed as percent of the probe-accessible area observed for a given amino acid in a given “standard state” conformation (e.g., as a monomeric helix or an extended conformation). Alternatively, the threshold could be expressed in Å² of area exposed to the probe.

One can also use the results of site-directed mutagenesis to define a set of sidechains that can be mutated with retention of biological function. These sidechains would be good candidates for water-solubilizing mutations.

Alternatively, residues that are likely to be exposed on the transmembrane surface of a protein are likely to have an environment (Zou, J. and Saven, J. G., J. Mol. Biol. 296:281-294 (2000); Kono, H. and Saven, J. G., J. Mol. Biol. 306:607-628 (2001)) that is strongly different from that expected for a water-soluble protein. These positions would also be potential targets for replacement in the third part of this process.

Alternatively, experimental methods such as photoaffinity labeling can be used to define the phospholipid-exposed positions. Variability in the nature of a residue at a given position in a set of phylogenetically or structurally related proteins has also been used to define membrane surface-accessible positions (Dieckmann, G. R. and DeGrado, W., Curr. Opin. Struct. Biol. 7:486-494 (1997)). Hydrophobicity profiles and moments have been used predict membrane-accessible surfaces in transmembrane proteins (Rees, D. C., et al., Science 245:510-513 (1989)). Membrane-accessible positions have distinct responses to site-directed mutagenesis, which can be used to predict surface-accessibility (Dieckmann, G. R. and DeGrado, W., Curr. Opin. Struct. Biol. 7:486-494 (1997)).

3) The next step is to use a computer program to search for combinations of amino acid sidechains that will provide water-solubility as well as conformational stability to maintain the desired 3-d structure. This program has three components a) a method to assign sidechains to positions identified in Kono, H. and Saven, J. G., J. Mol. Biol. 306:607-628 (2001); b) a potential function to evaluate the energies of the sidechains in these structures;

and c) a method to search through combinations of residues that provide a relatively low energy.

a) The potential function defines the approach used to define a sidechain at a given position. In one embodiment the sidechains of given residues are typically chosen from a set of all or some of the naturally occuring residues, and they are placed in low-energy conformation or rotamers. Energies are then computed using the potentials described below for all pairwise combinations. In a second embodiment, the sidechains are not actually built onto the backbone until a low-energy combination has been discovered using a simplified residue-based pairwise potential.

b) The energy is computed using a potential function, which can range from very simple to complex. In the simplest embodiment, the energies are scored based exclusively on the net charge of the amino acid sidechains and the distance of their C-beta atoms. Alternatively one could use sidechain-sidechain interaction pairwise potential functions in this step.

More complete potential functions consider the van der Waals potential, electrostatic interactions, hydrogen bonding, torsional energy, bond angles, bond lengths, and various “environmental energies” (Zou, J. and Saven, J. G., J. Mol. Biol. 296:281-294 (2000); Kono, H. and Saven, J. G., J. Mol. Biol. 306:607-628 (2001)) to account for solvation, hydrophobic effects and other factors that are difficult to express on a pairwise basis. Further, constants can be added for each amino acid type to account for differences in potential to adopt a given backbone conformation or as necessary correcting factors. A number of different methods have been shown to be useful for computing each these energetic terms and can be applied to computing water-solubilizing sequences (see Zou, J. and Saven, J. G., J. Mol. Biol. 296:281-294 (2000), Kono, H. and Saven, J. G., J. Mol. Biol. 306:607-628 (2001), and references cited within).

Constraints are included to assure that the surface residues have a charge and polarity consistent with water-solubility, or to encourage crystal contacts. This can be accomplished by requiring a given mean environmental score, by choosing a restricted set of polar amino acids, or by choosing a minimum number of polar, charged residues, or by specifying a threshold for the mean hydrophobicity of the atoms or residues on the surface of the protein.

3) Next one uses a search algorithm to define a collection of amino acids that will water-solubilize the protein while maintaining its three-dimensional structure. If only a few amino acids have been identified, it is possible to compute all possible combinations of sidechains. However, if the number of combinations is too large for this approach a number of methods can be employed which are well known to those skilled in the art. These include stochastic methods such as Monte Carlo algorithms and genetic algorithms, or deterministic methods such as dead end elimination, and other elimination methods (e.g., branch and bound).

See, for example:

1. Zou, J. and Saven, J. G., “Statistical theory of combinatorial libraries of folding proteins: energetic discrimination of a target structure,” J. Mol. Biol. 296:281-294 (2000).

2. Kono, H. and Saven, J. G., “Statistical theory for protein combinatorial libraries. Packing interactions, backbone flexibility, and the sequence variability of a main-chain structure,” J. Mol. Biol.306:607-628 (2001).

3. Dieckmann, G. R. and DeGrado, W. F., “Modeling transmembrane helical oligomers,” Curr. Opin. Struct. Biol. 7:486-494 (1997).

4. Rees, D. C., et al., “Hydrophobic organization of membrane proteins,” Science 245:510-513 (1989).

All of the documents referred to herein are incorporated by reference in their entireties.

Transmembrane proteins selected for water solubilization optionally include a binding site for at least one biologically active agent. The computationally designed mutated protein retains the binding site and the function of binding the biologically active agent.

These and other objects, advantages and features will become readily apparent in view of the following detailed description of the invention.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 gives the sequence of canine wild-type PLB as compared to several soluble mutants.

FIG. 2 shows a Circular Dichroism (CD) spectra of 125 μM water soluble Phospholamban (WSPLB) and residues 21 -52 of WSPLB.

FIG. 3A shows sedimentation equilibrium of WSPLB data fit to a monomer-pentamer equilibrium.

FIG. 3B shows sedimentation equilibrium of WSPLB data fit to a monomer-tetramer equilibrium.

FIG. 4A shows sedimentation equilibrium data of WSPLB (residues 21-52) fit to a monomer-tetramer equilibrium.

FIG. 4B shows sedimentation equilibrium data of WSPLB (residues 21-52) fit to a monomer-pentamer equilibrium.

FIG. 4C shows sedimentation equilibrium data of WSPLB (residues 21-52) fit to a monomer-tetramer-pentamer equilibrium.

FIG. 5 shows thermal denaturation of WSPLB.

FIG. 6 shows thermal denaturation of WSPLB and pWSPLB.

FIG. 7 shows thermal denaturation of WSPLB (residues 21-52).

FIG. 8 shows environmental “energy” Eₑₙᵥ vs. chain length for wild type KcsA (open circle) and the value Eₑₙᵥ that was used as a constraint in the sequence calculations (black circle).

FIG. 9A illustrates a molecular depiction of KcsA, wherein lipid-exposed residues of KcsA allowed to vary in the design are depicted along the inner and outer helices.

FIG. 9B illustrates a molecular depiction of KcsA with sidechains of mutated residues removed.

FIG. 9C illustrates a molecular depiction of WSK-3.

FIGS. 10A-D illustrate analytical gel filtration chromatography of WSK-1 (20 μM), WSK-1 (20μM, 6 M Urea), WSK-2 (100 μM), and WSK-3 (100 μM), respectively.

FIG. 11A shows equilibrium sedimentation analytical ultracentrifugation of 17 μM WSK-3.

FIG. 11B shows equilibrium sedimentation analytical ultracentrifugation of 17 μM AgTx₂-DNP in the presence and absence of 17 μM WSK-3 tetramer.

FIG. 11C shows equilibrium sedimentation analytical ultracentrifugation of BSA (36 μM) plus AgTx₂-DNP (17 μM).

FIG. 12 shows competition curves for binding of TEA to AgTx₂-DNP and WSK-3, and a second curve for binding of TMA, under similar conditions.

The present invention will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION OF THE INVENTION

### I. Phospholamban

In one embodiment, fully automated methods are developed, to introduce water-solubility to membrane helices, using the integral membrane protein phospholamban (PLB) as a model system for several reasons. First, it contains a single, helical membrane-spanning segment, and the entire protein is only 52 amino acids in length (Fujii, J., et al., Biochem. Biophys. Res. Comm. 138:1044-1050 (1986); Fujii, J., et al., J. Clin. Invest. 79:301-304 (1987)). The transmembrane region is proposed to form a structurally simple pentameric (Louis, C. F., et al., J. Biol. Chem. 257:5182-5186 (1982); Jones, L. R., et al., J. Biol. Chem. 260:7721-7730 (1985); Wegener, A. D., et al., J. Biol. Chem. 261:5154-5159 (1986); Watanabe, Y., et al., J. Biochem. 110:40-45 (1991)) coiled-coil (Simmerman, H. K. B., et al., J. Biol. Chem. 271:5941-5946 (1996)). Second, a large body of mutagenesis data exists to guide the choice of residues that can be safely mutated without compromising the structural integrity of the protein (Arkin, I. T., et al., EMBO J. 13 4757-4764 (1994); Simmerman, H. K. B., et al., J. Biol. Chem. 271:5941-5946 (1996); Kimura, Y., et al., J. Biol. Chem. 272:15061-15064 (1997)). Third, the biological importance of PLB makes structural information that might be gleaned from studying it particularly interesting. PLB is an integral membrane protein of cardiac sarcoplasmic reticulum, and is the primary downstream target of a phosphorylation cascade resulting from β-adrenergic stimulation. Its primary function is the regulation of the Ca²⁺-dependent ATPase SERCA2a (Vorherr, T., et al., Biochem. 31:371-376 (1992); Jones, L. R. and Field, L. J., J. Biol. Chem. 268:11486-11488 (1993); Toyofuku, T., et al., J. Biol. Chem. 269:22929-22932 (1994); Cornea, R. L., et al., Biochem. 36:2960-2967 (1997); Cornea, R. L., et al., J. Biol. Chem. 275:41487-41494 (2000)).

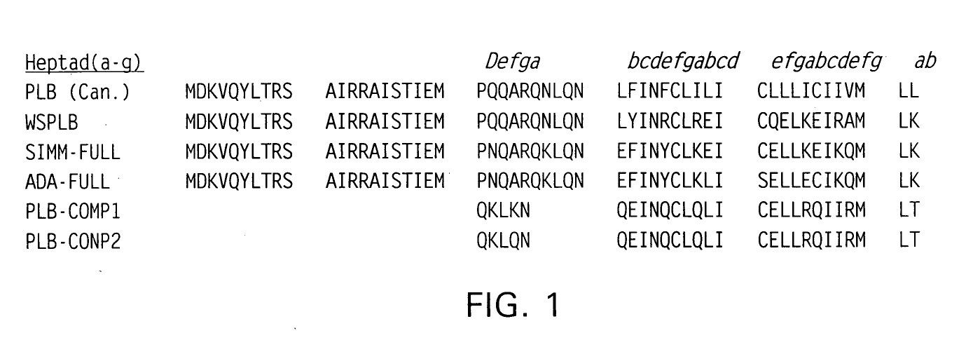

FIG. 1 illustrates the sequence of canine wild-type PLB as compared to several soluble mutants: WSPI-13, ADA-FULL, SIMM-FULL (Li, H., et al., Biochem. 40:6636-6645 (2001)), PLB-COMP-1 and PLB-COMP-2 (Sabine, F., et al., Biochem. 39:6825-6831 (2000)). In human PLI3, Asn27 is substituted by Lys. Positions S16 and T17 are phosphorylated by PKA and PKC. The differences between WSPLB and PLI3, the differences between SIMM-FULL and WSPLB, and the differences between PLB-COMP-1 (and 2) and SIMM-FULL are shown.

As shown in FIG. 1, PLB contains a cytosolic (residues 1-25) and transmembrane domain (residues 26-52) (Simmerman, H. K. B., et al., J. Biol. Chem. 261:3333-3341 (1986)), which together are 68-78% α-helical as determined by circular dichroism (CD). The transmembrane domain is about 73-82% α-helical in nondenaturing micelles composed of octylglucoside or C₁₂E₁₈, while in sodium dodecyl sulfate (SDS) micelles it is about 90% α-helical (Simmerman, H. K., et al., Biochim. Biophys. Acta 997:322-329 (1989)). Nuclear Magnetic Resonance (NMR) studies of the N-terminal peptide comprising residues 1-25 showed that it has little to no secondary structure in aqueous solutions (Terzi, E., et al., FEBS Letters 309:413-416 (1992); Hubbard, J. A., et al., J. Molec. Membrane Biol. 11:263-269 (1994); Mortishiresmith, R. J., et al., Biochem. 34:7603-7613 (1995); Quirk, P.G., et al., Eur. J. Biochem. 263:85-91 (1996); Li, M., et al., Biochem. 37:7869-7877 (1998)). These residues are not essential for pentamer formation and the transmembrane segment alone can form oligomers in detergent (Kovacs, R. J., et al., J. Biol. Chem. 263:18364-18368 (1988)).

Referring back to FIG. 1, PLB is phosphorylated on serine 16 and threonine 17 by cAMP-dependent protein kinase (PKA) and Ca²⁺-dependent protein kinase (PKC), respectively (Simmerman, H. K. B., et al., J. Biol. Chem. 271:5941-5946 (1996); Wegener, A. D., et al., J. Biol. Chem. 264:11468-11474 (1989)) following β-adrenergic stimulation. Phosphorylation increases the degree of association of PLB in SDS micelles and phospholipid bilayers, and also decreases its ability to activate SERCA2a (Wegener, A. D., et al., J. Biol. Chem. 264:11468-11474 (1989); Brittsan, A. G., et al., J. Biol. Chem. 275:12129-12135 (2000); Chu, G. X., et al., J. Biol. Chem. 275:38938-38943 (2000)). These observations suggest that it is the monomeric form of PLB that interacts with SERCA2a. The cytoplasmic region of PLB (1-25) is predominately positively charged (4 Arg, 1 Lys, 1 Asp, and 1 Glu) and phosphorylation of S16 and T17 changes the pI from 10-6.7 (Jones, L. R., et al., J. Biol. Chem. 260:7721-7730 (1985)). Thus, one model proposes that phosphorylation reduces the net positive charge on each monomer, relieving their electrostatic repulsion, and favoring pentamer formation.

All of the residues essential for pentamer formation are in the PLB transmembrane domain (Wegener, A. D., et al., J. Biol. Chem. 261:5154-5159 (1986)), determined in two mutagenesis studies using SDS-PAGE to monitor pentamer disruption (Arkin, I. T., et al., EMBO J. 13 4757-4764 (1994); Simmerman, H. K. B., et al., J. Biol. Chem. 271:5941-5946 (1996)). In both studies, amino acid positions in the transmembrane region were assumed to be in the protein core if their mutation disrupted the formation of PLB pentamers. Based on these data the PLB transmembrane domain was modeled as a left-handed coiled coil, containing L37, L44, L51, 140 and 147 in the apolar core with leucines at the “a” positions, and isoleucines at the “d” positions (Simmerman, H. K. B., et al., J. Biol. Chem. 271:5941-5946 (1996)).

Previously, two groups have produced water-soluble versions of PLB. Frank and coworkers (Frank, S., et al., Biochem. 39:6825-6831 (2000)) placed the core residues of PLB into a helix that contained the lipid-exposed residues of the water-soluble five-helix bundle cartilage oligomeric matrix protein (ICOMP) (PDB accession code 1vdf). This COMP-PLB hybrid, as shown in FIG. 1, exhibited poor solubility in water but, when fused with maltose binding protein, produced a protein that appeared to form pentamers and higher aggregates based on sedimentation velocity. Subsequently, Li and coworkers (Li, H., et al., Biochem. 40:6636-6645 (2001)) made two variants of a water-soluble PLB, SIMM-FULL and ADA-FULL, shown in FIG. 1, achieving better solubility and pentamer formation, but with dynamic properties reminiscent of a molten globule. Although these studies represent significant advances, it was difficult to assess the extent to which the water-soluble constructs had the same structure as native PLB.

Herein, a fully automated computational approach is disclosed for water-solubilizing membrane proteins, which are generally applicable to a variety of α-helical membrane proteins. In one embodiment, based on analysis of mutagenesis data, a model of the PLB homopentamer was computationally generated, and the exterior residues were redesigned to introduce water-solubility (WSPLB, MW=6293.4 Da). To address the structural similarity between PLB and WSPLB the effect of phosphorylation on the stability of WSPLB oligomers was demonstrated. The determinants of pentamer versus tetramer formation in WSPLB was also examined, and it was established that although full-length WSPLB peptides are uniquely in a monomer-pentamer equilibrium, a more stable heterogeneous mixture of tetramers and pentamers is present when the region encompassing residues 1-20 is removed. The ability to model, and predict the behavior of WSPLB upon either phosphorylation or truncation, reflects a similarity of its structure with PLB.

Design

Mutational data from Arkin and coworkers (Arkin, I. T., et al., EMBO J. 13 4757-4764 (1994)) and Simmerman and coworkers, (Simmerman, H. K. B., et al., J. Biol. Chem. 271:5941-5946 (1996)) was mapped into a numerical form, defining a “perturbation index” that ranges from 0 (a mutation which does not disrupt formation of the native pentamer) to 1 (a mutation which completely disrupts pentamer formation). An average perturbation index was calculated for each position in the PLB transmembrane domain and then graphed as a function of sequence position (Dieckmann, G. R. and DeGrado, W. F., Curr. Opin. Struct. Biol. 7:486-494 (1997)). The data were analyzed according to a sine wave describing the variation in the perturbation index (P) as a function of the position in the sequence (x):

P=a+b sin(2π(x+φ)/3.5 Eqn. 1

where a, b, and φ are fitting parameters, and the value of 3.5 residues is that of the left-handed coiled coil (Crick, F., Acta Crystallography 6:689-697 (1953)) shown for PLB (Arkin, I. T., et al., EMBO J. 13 4757-4764 (1994); Simmerman, H. K. B., et al., J. Biol. Chem. 271:5941-5946 (1996)). The value of φ is the phase, which defines the face of the helix that projects towards the interior of the structure. In agreement with Simmerman and coworkers (Simmerman, H. K. B., et al., J. Biol. Chem. 271:5941-5946 (1996)), this identifies the heptad repeat shown in FIG.1 in which Leu and Ile occupy the “a” and “d” positions respectively. Using this assignment a C₅ symmetric parallel left-handed coiled coil model was modeled for residues 31-52 using methods described previously (North, B., et al., J. Molec. Biol. 311:1081-1090 (2001)). This orientation is similar to that seen in the crystal structure of COMP (Malashkevich, V. N., et al., Science 274:761-765 (1996)) a soluble pentameric coiled coil, and also seen in the original model of Simmerman and coworkers (Simmerman, H. K. B., et al., J. Biol. Chem. 271:5941-5946 (1996)), and a more recent model of Arkin and coworkers (Torres, J., et al., J. Mol. Biol. 300:67785 (2000)).

Based on this model, it was determined that lipid-exposed residues F32, F35, 138, L39, L42, L43, 145, C46, 148, V49, and L52 could be mutated without disturbing the structural integrity of the bundle. These residues showed minimal contact with neighboring helices. The sequence design began by choosing Tyr as a replacement for Phe 32 providing a chromophore that could be used for concentration measurements, as well as aiding solubility while minimizing sequence perturbations.

An amino acid based approach—rather than an atom-based approach—was chosen for the design of the lipid-exposed residues because of the difficulties in specifying unique orientations for lipid-exposed side chains. Side chains of surface residues tend to be highly mobile (Zhao, S., et al., Proteins 43:271-279 (2001)) and often adopt multiple conformations in solution. The search for a single rotamer/sequence combination for these lipid-exposed residues, therefore, was abandoned for a more general residue-centric approach. The use of amino acid pairwise potential should be analogous to calculating the mean-field energy between residues (Lee, C. and Subblah, S., J. Mol. Biol. 217:373-388 (1991)).

The lipid-exposed residues were chosen using a computer program, which was designed to minimize the residue-based energy function of the entire transmembrane sequence. Following a Monte-Carlo/simulated annealing approach, the program was used to optimize the remaining 10 variable amino acids against the background of the fixed (core and non-helix spanning) residues. The energy function used to score sequences is described below and was chosen to optimize both intra- and inter-helical interactions, as well as produce a sequence that was hydrophilic enough to be water-soluble. This algorithm resulted in the selection of the sequence of WSPLB that was expressed in E. coli. This protein was phosphorylated at S16 with cAMP-dependent protein kinase providing pWSPLB. Finally, a peptide corresponding to residues 21-52 was synthesized, denoted WSPLB (21-52).

In mutating the transmembrane proteins, native residues or side chains can be replaced by both naturally occurring and non-naturally occurring residues or side chains. A residue or side chain can be replaced by a more hydrophilic or more hydrophobic residue, as long as the resulting mutated protein is water soluble. An example of a mutation that results in placing a more hydrophilic residue or side chain into the sequence would be the replacement of alanine with aspartic acid.

CD Spectroscopy

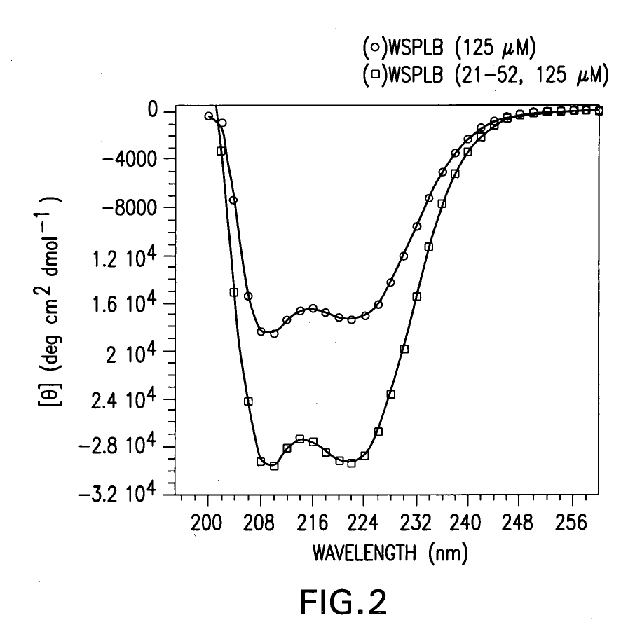

Circular dichroism (CD) spectroscopy was used to examine the secondary structure of WSPLB. FIG. 2 shows a Circular Dichroism (CD) spectra of 125 μM WSPLB and W9P LB (residues 21-52). WSPLB spectra taken in 10 mM sodium phosphate pH 7.5, 50 mM NaCl, 1 mM TCEP-HCl. WSPLB (residues 21-52) spectra taken in 15 mM MOPS pH 7.0, 50 mM NaCl, 1 mM EDTA, and 1 mM TCEP-HCl. The spectra are concentration dependent as would be expected for a self-associating peptide. However, as shown in FIG. 2, at concentrations greater than approximately 100 μM, the spectra are essentially independent of concentration, and show a double minimum at 208 and 222 nm, the hallmarks of the α-helix. At 125 μM the ellipticity at 222 nm [θ₂₂₂] is −17,400 deg cm² dmol⁻¹, which is similar to the range of values (−20,000 to −25,000 cm² dmol⁻¹) observed for the full length native PLB in DMPC vesicles (Arkin, I. T., et al., J. Molec. Biol. 248:824-834 (1995)) and C₁₂E₈, and octyl glucoside detergents (Simmerman, H. K., et al., Biochim. Biophys. Acta 997:322-329 (1989)). Also, at similar protein concentrations, the spectrum of the phosphorylated form pWSPLB was the same within experimental error, suggesting that the secondary structures of the proteins are similar. Analysis of the spectra of WSPLB indicate that approximately 25 residues are in an α-helical conformation (Chakrabartty, A., et al., Nature 351:586-588 (1991)), corresponding to the transmembrane helical region. The similarity of the spectra of WSPLB and native PLB suggest that they may have similar overall structures, with a disordered amino terminal segment followed by a α-gelical coiled coil within the C-terminal half of the protein.

To provide additional support for this suggestion, the CD spectrum of the truncated peptide WSPLB (21-52) was examined, as shown in FIG. 2. The helicity calculated for this peptide is approximately 85% (Chakrabartty, A., et al., Nature 351:586-588 (1991)) ([θ₂₂₂] =−29,453 deg cm² dmol⁻¹), again corresponding to about 25 residues in a helix.

Oligomeric state of WSPLB

Size exclusion chromatography was used as an initial screen of oligomeric state and homogeneity of both peptides. At two loading concentrations (460 and 45 μM) WSPLB eluted as a single symmetrical peak from a G75 Superose column previously calibrated with globular molecular weight standards (data not shown). The slope of the peak suggested that it formed a single molecular species. The observed mass for WSPLB was 23,500+/−4000 versus a calculated monomer mass of 6294. Deviations of WSPLB molecular weights from those expected are, for the pentameric helical bundle, assumed to be due to the non-globular structure of helical bundles. The truncated WSPLB (residues 21-52) peptide was run at two concentrations with and without boiling. In this case, a concentration dependent mixture of two species eluting at 22 ml and 31 ml (MWₐₚₚ 16000 and 11500 respectively) was observed, versus a calculated monomer mass of 3956. The mass difference between these two species observed is 4520, roughly one monomer. Thus, two associating species need to be considered.

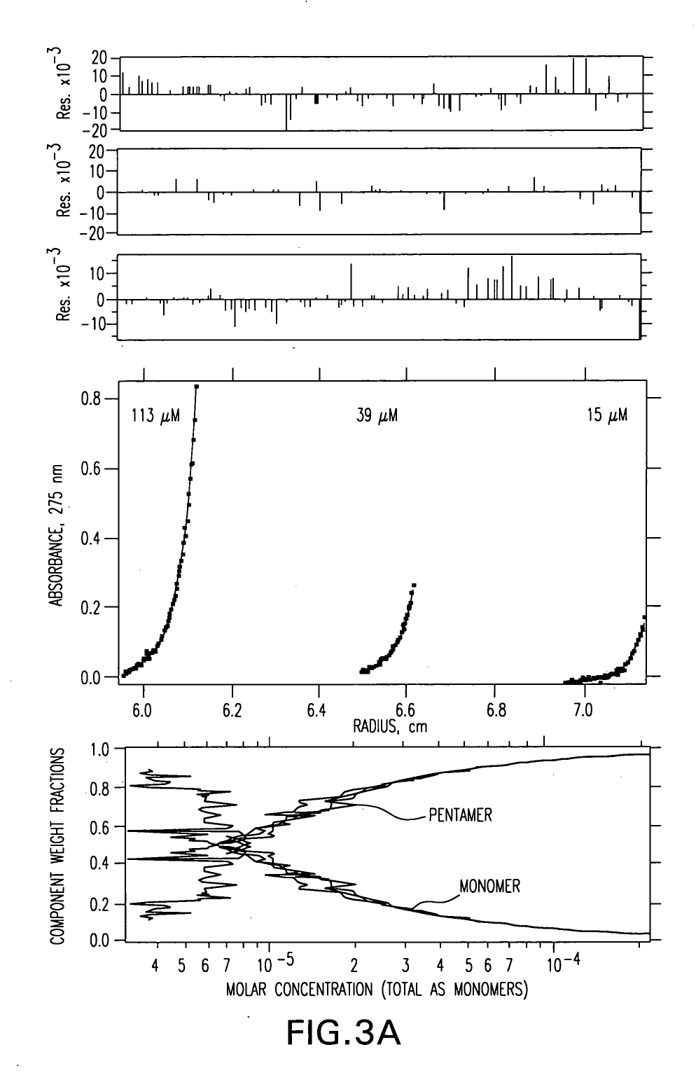

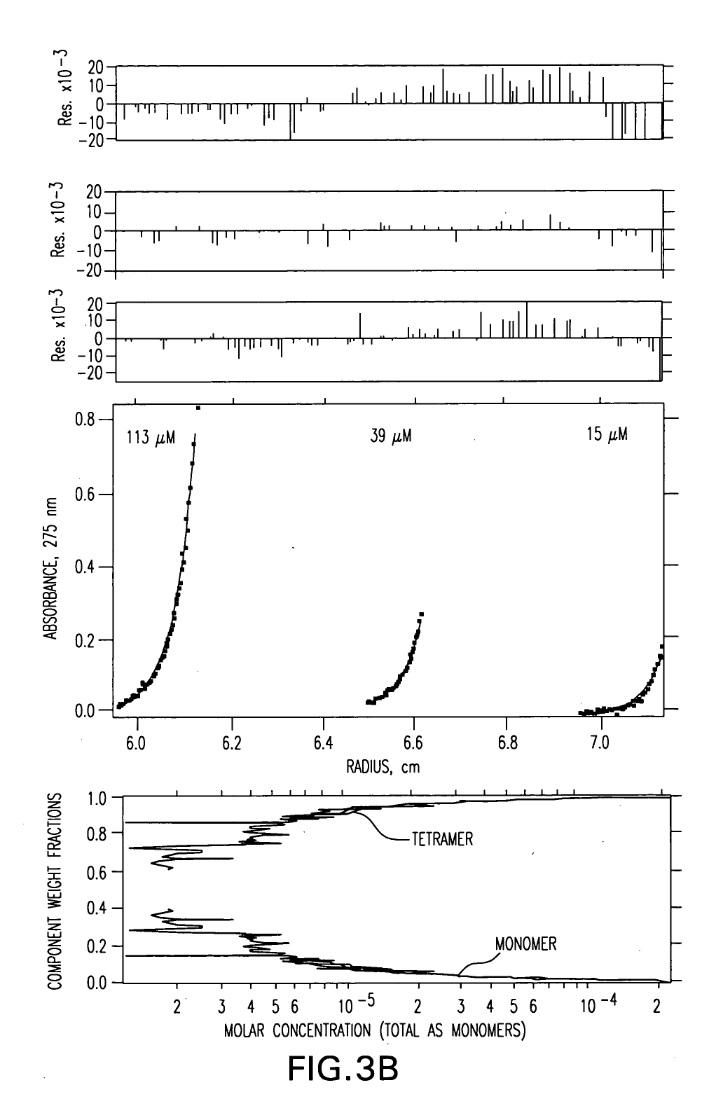

Sedimentation equilibrium was used to rigorously determine the association state and thermodynamics of the observed oligomers. The results are shown in FIGS. 3A-B and 4A-C. Because WSPLB eluted as a single homogenous species from size exclusion chromatography, and its CD spectra were concentration independent above 100 μM, we initially determined its association state at 113 μM centrifuged at 35,000 rpm. In these conditions, WSPLB sedimented as a single species with a molecular weight of approximately 31,600+/−200, or a pentamer (n=5.04). Because CD spectra showed a concentration dependence below 50 μM, peptide samples of 113,39 and 15 μM were then centrifuged at 30000, 35000, and 40,000 rpm, and the nine data sets were used to globally determine the monomer-pentamer dissociation constant, as shown in FIG. 3A. The monomer-pentamer dissociation constant was determined to be 6.34×10⁻²¹ M⁴ with a P_{(1/2)}=6 μM, where P_{(1/2)} is the midpoint of the monomer-nmer isotherm in total peptide concentration.

Sedimentation equilibrium was also used to determine the molecular weights of species present in the truncated WSPLB (residues 21-52). WSPLB (residues 21-52) (97 μM initial loading concentration) was centrifuged at 43,000 rpm, and a single species fit yielded a molecular weight of −17,300 (n=4.4) a molecular weight intermediate between tetramers and pentamers.

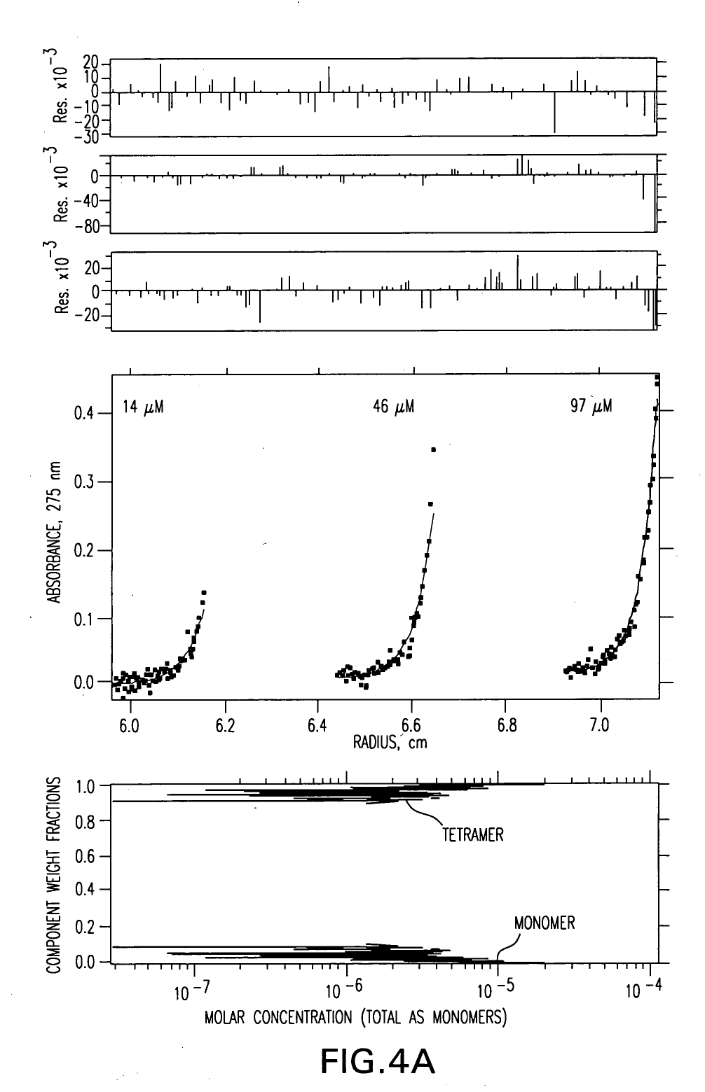

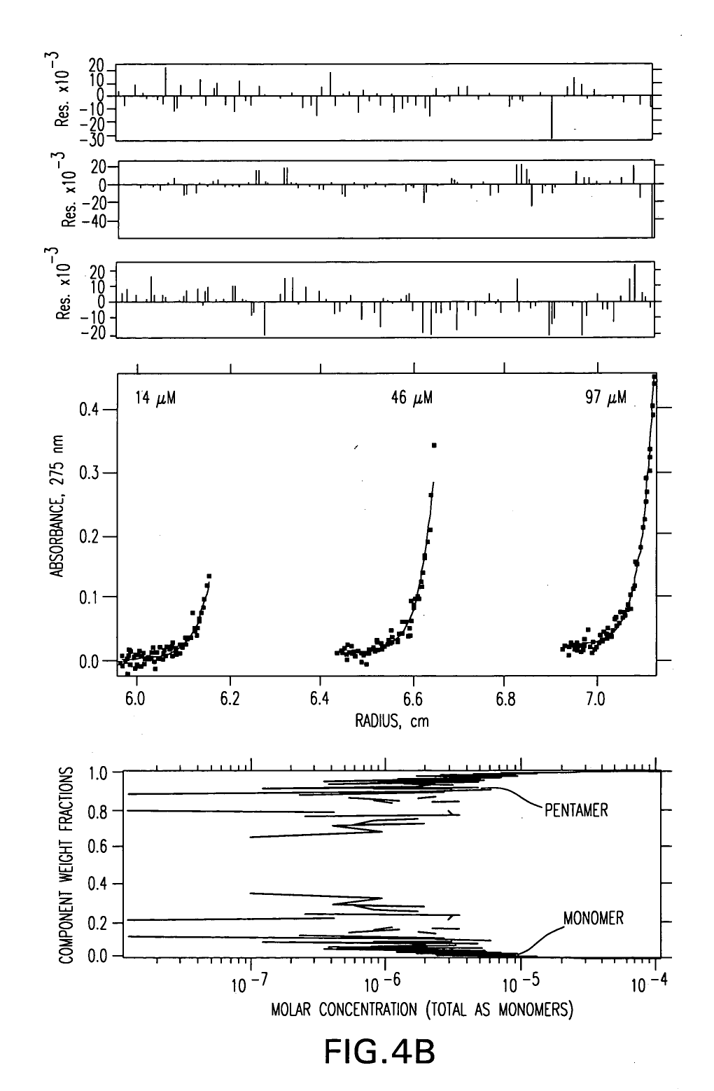

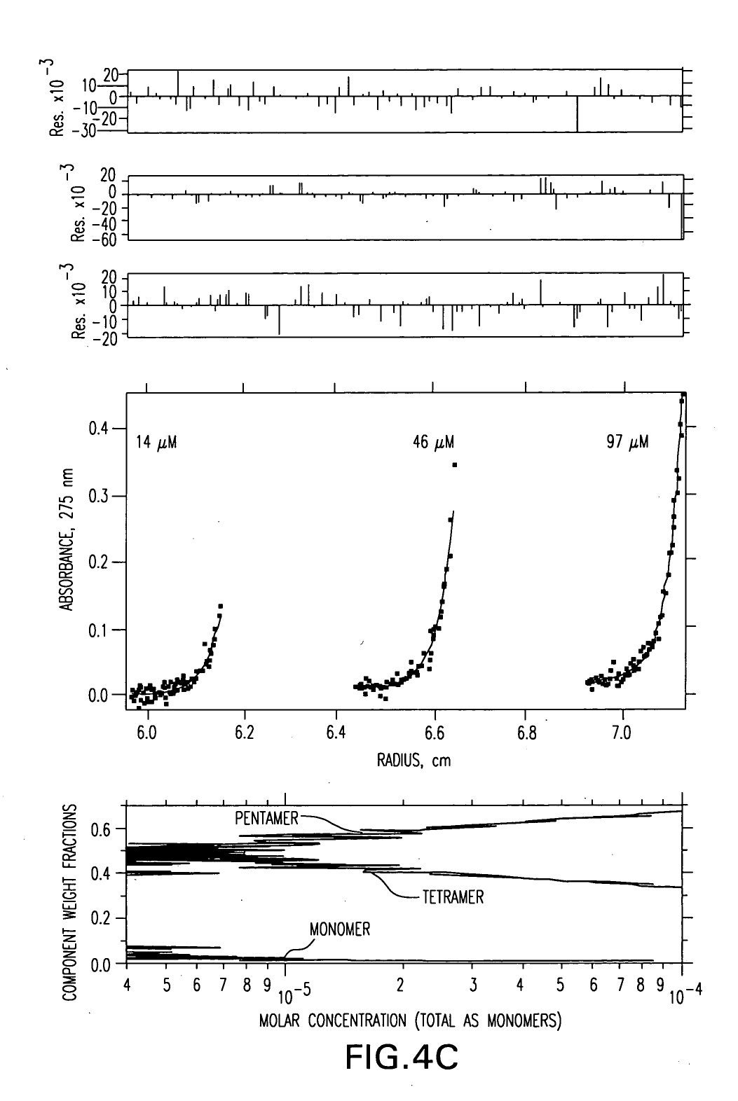

Subsequently, two lower concentrations (14 and 46 μM) of this peptide were also centrifuged, at 43,000 rpm, and all three data sets were used to determine the monomer-nmer dissociation constant. Various equilibrium models were used to fit the parameters including monomer-tetramer, as shown in FIG. 4A, monomer-pentamer, as shown in FIG. 4B, and monomer-tetramer-pentamer equilibrium, as shown in FIG. 4C. The best fit was to a monomer-tetramer-pentamer. The monomer-tetramer-pentamer fit yielded a monomer-tetramer dissociation constant of 1×10⁻²¹ M³ and an overall monomer-pentamer dissociation constant of 1×10⁻²⁸ M⁴.

Although all three schemes provide a good fit to the data, the presence of multiple peaks on size exclusion chromatography requires the use of a monomer-tetramer-pentamer scheme.

Stability of WSPLB constructs

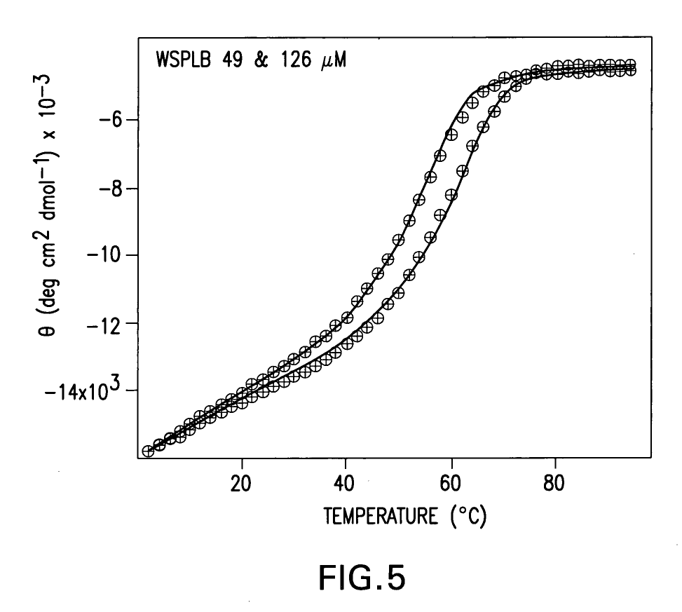

All of the peptides were thermally unfolded, monitoring the loss of signal at 222 nm by CD spectroscopy with increasing temperature. Scans taken with 60 s signal averaging time and equilibration of 4 min at each temperature. Concentrations of peptide are 50 and 125 μM. Data were fit to a two-state model (theoretical curves are shown) with a monomer-pentamer equilibrium. FIG. 5 shows the unfolding curve of WSPLB including a single transition whose midpoint depends on concentration, as expected for a monomer-oligomer equilibrium. Previously it has been shown that global fitting of such curves obtained at multiple protein concentrations can be used to confirm the aggregation state and obtain highly accurate measures of the free energy of association. Application of these procedures to WSPLB allows an excellent fit for a monomer-pentamer equilibrium, providing the thermodynamic parameters described in Table 1. The predicted midpoint concentration for the monomer-pentamer isotherm was 4.9 μM (30 kcal/mol) at 25° C., in good agreement with the value of 6 μM (28 kcal/mol) obtained from sedimentation equilibrium.

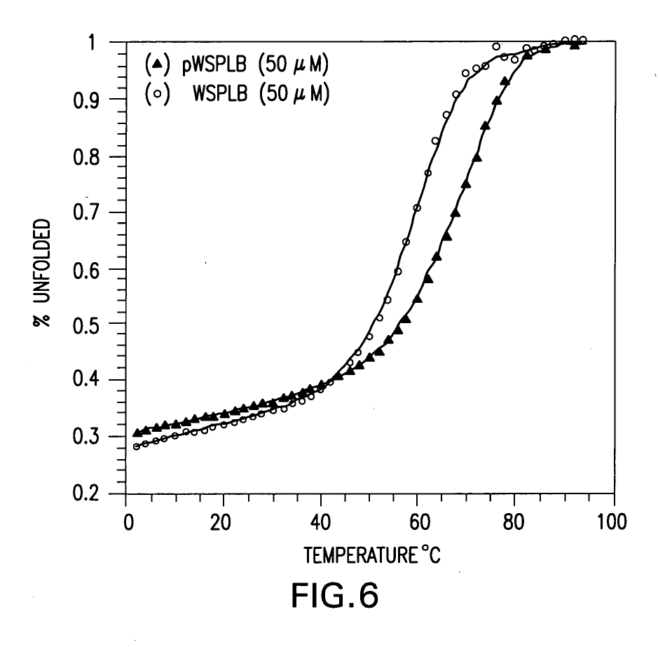

FIG. 6 shows thermal denaturation of phosphorylated WSPLB (PWSPLB) from 2 to 94° C., normalized to % unfolded peptide. Conditions are identical to those in FIG. 2. Scans taken with 60 s signal averaging time and equilibration of 4 min at each temperature. Concentrations of both peptides are 50 μM. As shown in FIG. 6 and Table 1, thermal denaturation showed that phosphorylation of Ser16 significantly increased the stability of the pentamer. At these concentrations the ΔTₘ was approximately 8° C., and the ΔΔG_{unf} was 4 kcal/mol. In Table 1, parameters are shown for full length WSPLB and phosphorylated pWSPLB, including ΔG_{unf}, ΔH_{unf}, and ΔCp, all at 1 M standard state, and a reference temperature of 350 K. Tₘ at 50 μM is also shown. ACP was a global fitting parameter for WSPLB, but held fixed for pWSPLB at the same value as WSPLB.

TABLE 1
Thermodynamic parameters derived from global fitting of thermal
denaturation curves as measured by circular dichroism (CD).

| | Tₘ at 50 μM | ΔG_{unf} | ΔH_{unf} | ΔCp |
|---|---|---|---|---|
| Construct | (° C.) | (kcal/mol) | (kcal/mol) | (kcal/mol) |
| WSPLB | 55 | 19.2 | 229 | 1.9 |
| PWSPLB | 63 | 23.1 | 231 | 1.9 |

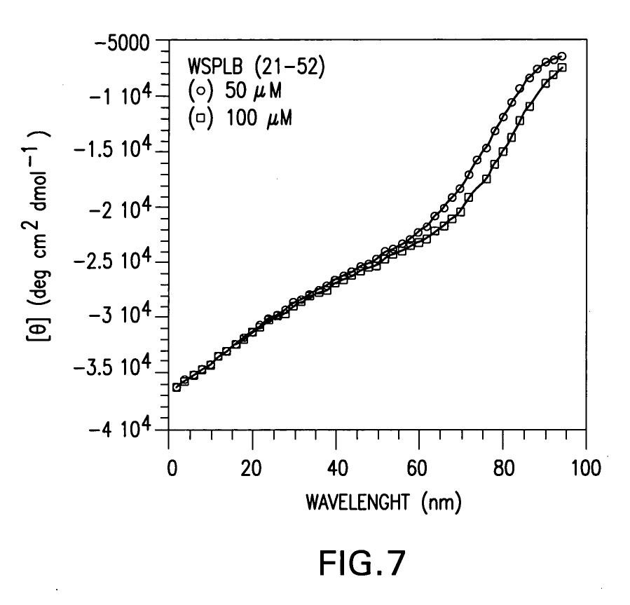

Thermal unfolding curves of WSPLB (residues 21-52) from 2 to 94° C. are illustrated in FIG. 7. Scans taken with 60 s signal averaging time and equilibration of 4 minutes at each temperature. Conditions and peptide concentrations were identical to those in FIG. 2. Unlike WSPLB, this peptide showed a pre-transition at low temperature, which was independent of peptide concentration. At higher temperatures a main transition is observed, which depends on the concentration of the peptide. Because the first transition has a small amplitude and does not appreciably depend on concentration, it may correspond to a switch between tetramer and pentamer aggregation states which should show a very weak concentration dependence. In contrast, the main transition corresponds to dissociation of the oligomer to unfolded monomer. The increase in stability of WSPLB (residues 21-52) to WSPLB, also seen in sedimentation equilibrium, is apparent in the increase in Tₘ which is 76° C. for WSPLB (residues 21-52) versus 55° C. for WSPLB at 50 μM.

Phospholamban Discussion

To produce a water-soluble variant of PLB, the transmembrane lipid-exposed residues have been computationally mutated from native, hydrophobic, amino acids to polar amino acids. In the absence of a 3-dimensional structure of the PLB pentamer, indirect methods were used to infer which of the transmembrane residues are lipid exposed. In the case of PLB, a large amount of mutagenesis data has been amassed by other groups (Arkin, I. T., et al., EMBO J. 13 4757-4764 (1994); Simmerman, H. K. B., et al., J. Biol. Chem. 271:5941-5946 (1996); Kimura, Y., et al., J. Biol. Chem. 272:15061-15064 (1997)), which was used to guide the building of a structural model of the pentamer, allowing us to choose those amino acids which might be mutated to introduce water-solubility.

Although a great deal of information is known about the membrane protein PLB, there remain several key pieces of information that have not been obtained until now. The effect of phosphorylation on WSPLB oligomerization has been studied by SDS-PAGE (Wegener, A. D, and Jones, L. R., J. Biol. Chem. 259:1834-1841 (1984); Wegener, A. D., et al., J. Biol. Chem. 264:11468-11474 (1989); Arkin, I. T., et al., EMBO J. 13 4757-4764 (1994); Reddy, L. G., et al., J. Biol. Chem. 270:9390-9397 (1995)), electron paramagnetic resonance (Cornea, R. L., et al., Biochem. 36:2960-2967 (1997)), and on the catalytic activity of SERCA2a (Sham, J. S. K., et al., Am. J. Physiol. 261:H1344-H1349 (1991); Cantilina, T., et al., J. Biol. Chem. 268:17018-17025 (1993)) but its energetic contribution to PLB pentamer stabilization has heretofore not been measured. Phosphorylation at Ser16 and Thr17 has been suggested to have several effects on the system. It may decrease the electrostatic repulsion between positively charged monomers (Jones, L. R., et al., J. Biol. Chem. 260:7721-7730 (1985)), or destabilize the interaction of PLB with SERCA2a as well as with the negatively charged membrane (Plank, B., et al., Eur. J. Biochem. 136:215-221 (1983); Tada, M. and Inui, M., J. Mol. Cell. Cardiol. 15:565-575 (1983); Inui, N. L., et al., J. Biol. Chem. (261):1794-1800 (1986); Kirchberger, M. A., et al., Biochem. 25:5484-5492 (1986); Suzuki, T. and Wang, J. H., J. Biol. Chem. 261:7018-7023 (1986)), shifting the monomer-pentamer equilibrium toward the pentamer. The results disclosed herein show that phosphorylation increases the stability of WSPLB by -0.9 kcal/mol/monomer, demonstrating that some part of the role of phosphorylation is exclusively an effect on PLB itself. The fact that phosphorylation has a similar effect on both PLB and WSPLB suggests also that the core and interhelical packing interactions are maintained in the design disclosed herein.

Both size exclusion chromatography in SDS followed by laser light scattering (Watanabe, Y., et al., J. Biochem. 110:40-45 (1991)), and sucrose density centrifugation with octyl glucoside (Harrer, J. M. and Kranias, E. G., Molec. Cell. Biol. 140:185-193 (1994)), show that phospholamban is a mix of mainly monomers and pentamers in the membrane. Studies disclosed herein show that WSPLB exists in a monomer-pentamer equilibrium when the protein is full length. However, when the regulatory region of phosphorylation is removed (residues 1-20), the oligomeric state is actually stabilized, with the production of a significant population of tetramers. Although the full-length WSPLB peptide is more specifically a pentamer, it is less stable than WSPLB (residues 21-52). Thus, the region (residues 1-20) of WSPLB acts as a negative design element, destabilizing any oligomer formed, but specifying pentamer over tetramer in the full-length peptide. Trading stability for specificity by burying polar sidechains has been seen in other model peptides (Hill, R B. and DeGrado, W. F., J. Am. Chem. Soc. 120:1138-1145 (1998); Hill, R. B., et al., J. Am. Chem. Soc. 122:746-747 (1999); Hill, R. B., and DeGrado, W. F., Structure, Folding, and Design 8:471-479 (2000); Lumb, K. J. and Kim, P. S., Biochem. 37:10342 (1998)) and suggests that the presence of the PLB cytoplasmic domain allows intermolecular interactions within the oligomer to favor pentamers.

The propensity for tetramers in WSPLB, and possibly PLB, is consistent with previous results on the model system GCN4-pLI (Harbury, P. B., et al., Science 262:1401-1407 (1993)). This tetrameric peptide contains the same hydrophobic core repeat as PLB, with salt bridges between residues at the “e” and “g” positions. It is possible that the nature of the residues at these “e” and “g” positions influence the aggregation state. For example, in PLB and WSPLB, Q29 (a “g” position) and N34 (an e position) might form an interhelical interaction analogous to a hydrogen bond between N41 and E36′ in the 5-helix bundle COMP (Malashkevich, V. N., et al., Science 274:761-765 (1996)). Also, if the helical bundle in PLB extends beyond the hydrophobic transmembrane region, Q26 and N30 would occupy buried “d” and “a” positions. In the crystal structure of COMP (Malashkevich, V. N., et al., Science 274:761-765 (1996)) Gln54 is buried in the hydrophobic core at a “d” position.

Studies disclosed herein present the first experimentally verified, fully automated design procedure for introducing water-solubility to membrane-spanning α-gelical proteins. In one embodiment, using the model system PLB, the determinants of WSPLB oligomerization have been studied, and the effect of phosphorylation has been quantified on its stability. By making the PLB transmembrane helix water-soluble, a system was designed, which has a similar structure and behavior as membrane soluble PLB, and may contain all of its essential features. Although PLB and WSPLB have different solubilities, and therefore different forces stabilizing their folded states, both proteins showed the same oligomerization and response to phosphorylation. With a thermodynamic characterization of PLB, it may be found that the absolute values of stabilities measured of WSPLB may be different from PLB because of the introduction of the hydrophobic effect upon water-solubilization. However, it has been shown herein, that the process of water-solubilization of certain membrane-spanning helices will not alter their global properties and structure.

Phospholamban Protein Design

The energy function used in the PLB protein design can be written as the sum of the energy due to intrinsic helical propensities of the amino acids, the intrahelical pairwise residue interaction energies, the interaction energy between the residues and helix macrodipole, the interhelical electrostatic interaction energy, a “solubility” term to enforce a low overall hydrophobicity, and a sequence entropy term as follows:

$$\begin{matrix}E = {ω}_{helix} {\sum }_{i = 1}^{{N}_{m}} {E}_{i}^{helix} + {ω}_{AGADIR} {\sum }_{i = 1}^{{N}_{m}} {\sum }_{j = 1}^{{N}_{m}} {E}_{i , j}^{AGADIR} + \\ {ω}_{macrodipole} {\sum }_{i = 1}^{{N}_{m}} {E}_{i}^{macrodipole} + {ω}_{interhelix} {E}^{interhelix} + \\ {ω}_{solubility} {E}^{solubility} + {ω}_{sequence} {E}^{sequence}\end{matrix} \qquad Eqn . 2$$

Each term has weight (ω) that can be used to tune the relative strengths of the energy terms. Each weighting term has been set to 1.0 unless otherwise noted. The first term in Equation 2 is the α-helix partition energy, taken from the analysis of helical propensities of O'Neil and DeGrado (O'Neil, K. T. and DeGrado, W. F., Science 250:646-651 (1990)). Since one of our goals was to maintain the helical nature of the transmembrane helices of phospholamban, this term was applied such that amino acids with higher α-helical propensity should contribute favorably to the energy term.

The second term in Equation 2 represents intrahelical i to i+3 and i to i+4 interaction energies. The values used to represent these energies were taken from the program AGADIR (Mufioz, V. and Serrano, L., Nature 1:399-409 (1994)). This set of intrahelical interaction energies was originally derived to predict the percent helicity of a peptide of a given sequence, but here we are using the interaction energies as a function that can be searched in order to find a sequence with an optimal energy (Villegas, V., et al., Folding & Design 1:29-34 (1996)). An update to AGADIR (Muñoz, V. and Serrano, L., J. Molec. Biol. 245:275-296 (1995)) contains a term that accounts for interaction of charged residues with the helix macrodipole, represented by the third term in Equation 2.

To measure interhelical electrostatic interactions, amino acids were assigned a designation of either “+” or “−” according to their charge at neutral pH, and interacting pairs were scored as previously reported (Summa, C. M., et al., “Computational de novo design, and Characterization of an A₂B₂ Diiron Protein.” J. Mol. Biol.321:923-938 (2002)). These scores are a simplified representation of experimentally measured interhelical electrostatic interaction energies (Krylov, D., et al., EMBO J. 13:2849-2861 (1994)). Since the energies were intended only as an approximation to find an optimal pattern of charge, and not to calculate binding energies, this simplification was justified. All e-g′ (an interaction between the e residue of a given helix, and the nearest g residues of a neighboring helix), b-g′, e-c′ and b-c′ pairs were assumed to be interacting. These interactions were summed over all contacts i giving the fourth term in Equation 2 as follows:

$${E}^{interhelix} = {\sum }_{i = 1}^{{N}_{contact}} {E}_{contact} ( i ) \qquad Eqn . 3$$

An upper limit was enforced on the hydrophobicity of the coiled coil region by adding a term that penalized sequences that have a hydrophobicity that is greater than that of the water soluble pentameric peptide, COMP. The average per-residue hydrophobicity of the COMP structure for the sequence analogous to the transmembrane domain was calculated to be 0.372 kcal/mol (using the octanol-water transfer free energies of the amino acids) (Fauchere, J.-L. and Pliska, V., Eur, J. Med. Chem. 18:369-375 (1983)). If the per-residue hydrophobicity of any sequence exceeded this value, then the solubility energy term was defined as:

S^{sequence}=−1n(W) Eqn. 4

Otherwise, this parameter was set to a value of 0.0. This has the effect of preventing all sequences with a higher hydrophobicity score than the COMP sequence from appearing in the optimal sequence set.

In order to force sequence diversity, a term was added called the “sequence entropy” which has been defined as follows:

E_{solubility}=(h−0.372)*10 Eqn. 5

where

$$W = \frac{( {\sum }_{i = 1}^{20} {N}_{i} )}{{\prod }_{i = 1}^{20} ( {N}_{i} ! )} \qquad Eqn . 6$$

Nᵢ represents the number of residues in the full sequence with an amino acid identity of type i. In the calculations below, this term was given a scaling factor of 0.1 so that its absolute value was roughly equal to those of the other terms in Equation 2.

The sequence was optimized using a Monte-Carlo/simulation annealing algorithm run from 700° K to 10 K with linear decrements over 700000 steps. This process was repeated 500 times and the sequences were then ranked and analyzed. For each energy calculation the entire sequence (the variable residues as well as the non-variable residues) of the transmembrane domain was considered in the calculation. The top scoring sequence was built onto the backbone structure and analyzed. Amino acid side-chains were modeled on an SGI Indigo2 workstation running the program InsightII (Molecular Simulations, Inc., San Diego, Calif.). The sequence that was eventually produced differs slightly from the automatically designed sequence because of steric clashes that could not have been predicted with a residue-based energy function. This highlights the need for consideration of atomic level detail in protein design algorithms, but does not diminish the usefulness of residue-based functions for initial screening of possible sequences.

Modeling of Perturbation Index Data

The parameters were fit to the data on an Apple PowerBook G3 using the program Kahedagraph (Synergy software). The data points for residue numbers greater than 49 were not evaluated; data for these residues seemed to contradict pentamer sensitivity data from another study (Simmerman, H. K. B., et al., J. Biol. Chem. 271:5941-5946 (1996)) and were therefore excluded.

Expression and Purification of WSPLB.

The gene for WSPLB (MW=6293.4) was synthesized by PCR overlap extension using four primers. This gene product was cloned at BamHI and EcoRI sites into HT-UK vector (Gregory VanDuyne lab), a variant of pET2 1 a (Novagen) which contains a tobacco etch virus (TEV) protease cleavage site N-terminal to a six-histidine tag. The protein was expressed in BL21 (DE3) cells (Novagen) in terrific broth for four hours after induction at OD 0.6 with 0.5 mM isopropyl-β-D-thiogalactoside. After expression, cells were harvested at 4° C. by centrifugation at 5000 rpm. Cells pellets were lysed in denaturing lysis buffer by resuspension in 6M Gdn/GdnHCl, 0.1 M sodium phosphate, 0.01 M Tris-HCl pH 8.0, and stirring for 3 hours. This mixture was then sonicated and centrifuged at 13,000 rpm in preparation for purification.

For purification, the lysed cells were loaded onto a 25 ml Ni²⁺ Superose (Quiagen) column in the lysis buffer. The column was washed with lysis buffer, previously adjusted to pH 6.3. WSPLB-6His was eluted isochratically with the same buffer adjusted to pH 4.5, plus 1 mM imidazole. Fractions of the eluate were collected, and the presence of WSPLB-6His was verified by gel electrophoresis on a 12% Bis-Tris SDS-PAGE reducing gel (Invitrogen). Fractions containing WSPLB-6His were then pooled, concentrated, and diluted with TEV protease cleavage buffer to a final component mixture of 200 mM NaCl, 1 M Gdn/GdnHCl, 0.1 M sodium phosphate buffer, 0.01 M Tris-HCl pH 8.0, 1 mM ethylenediaminetetraacetic acid EDTA) and 1 mM dithiothreitol (DTT). Cleavage with 2000 U TEV protease (Life Technologies) proceeded in this buffer for 3 days at 30° C., until −80% of the peptide was cleaved. Finally, WSPLB was purified by reverse-phase HPLC on a Vydac C4 preparative column using a linear gradient of water and acetonitrile containing 0.1% trifluoroacetic acid (TFA). Purity was assessed by analytical reverse-phase HPLC, MALDI-TOF mass spectrometry, and 12% Bis-Tris reducing SDS-PAGE gels.

Peptide synthesis of WSPLB (residues 21-52)

WSPLB (residues 21-52, MW. 3956) was chemically synthesized as a C-terminal carboxyamide on a 0.25 mmol scale using an Applied Biosystems model 433A solid phase peptide synthesizer (Perkin-Elmer) with standard FMOC amino acid chemistry. Peptides were washed on the resin with DMF and ether, cleaved for 1.5 hours with TFA:water:thioanisole:ethane-dithiol:phenol (40/2/2/1/3 v/v/v/v/w) and subsequently precipitated with cold ether. Purification proceeded by reverse-phase HPLC using a preparative C4 column (Vydac) and a linear gradient of the appropriate buffers. The purity of samples was then verified by MALDI-TOF mass spectrometry, analytical reverse-phase HPLC and 12% Bis-Tris reducing SDS-PAGE gels.

Phosphorylation of WSPLB

Expressed and purified WSPLB was phosphorylated enzymatically using cAMP-dependent protein kinase (PKA) catalytic subunit (New England Biologicals). To determine the efficiency of phosphorylation of WSPLB, initial screens using ATP Y³² P were conducted containing 10 μM WSPLB, 1 MM ATP, 16.6 nM ATP Y³² P, 5 U PKA, 150 mM NaCl, 50 mM Tris-HCl pH 7.5 and 10 mM MgCl₂. The reaction was allowed to proceed for 1.5 hours at 30° C., and was separated from reaction contents by gel electrophoresis using 12% Bis-Tris reducing SDS-PAGE. After drying the gel, the extent of phosphorylation was visualized by exposure on a Molecular Dynamics Storm 280, and analyzed using Image Quant v1.11 software. The extent phosphorylation was determined to be greater than 90% (data not shown). The reaction was repeated on a preparative scale with 334 μM WSPLB, 3 mM ATP, and 15 U PKA at 30° C. Phosphorylated protein was separated from the reaction contents by gel filtration chromatography using G-25 resin, and monitored at 280 nm.

Circular dichroism spectroscopy

All CD spectra were collected on an AVIV 62DS spectropolarimeter, using a 1 mm pathlength quartz cuvette. CD spectra of WSPLB and pWSPLB were collected in 10 mM sodium phosphate pH 7.5, 50 mM NaCl, 1 mM tris (2-carboxyethyl)-phosphine hydrochloride (TCEP-HCl), and 1 mM EDTA while those for WSPLB (residues 21-52) in the same buffer substituted with 50 mM MOPS pH 7. Each wavelength scan from 200-260 nm is an average of four scans with 4 second averaging time per data point at 25° C. All thermal melting curves were collected in the same buffers as their corresponding wavelength scans, with 60 second averaging time and 4 minute equilibration time. The elipticity was measured at 222 nm as a function of increasing temperature (2-94° C.). WSPLB and pWSPLB melting curves were analyzed with Igor Pro® 3.16 (available from WaveMetrics, Inc., Oswego, Oreg. 97035), assuming a two-state unfolding pathway. These data were treated as a monomer-nmer two-state system, using the functional form of the Gibbs-Helmholtz formula described previously (Boice, J. A., et al., Biochem. 35:14480-14485 (1996)):

$$\begin{matrix}[ θ ] = {[ θ ]}_{mon} \frac{[ mon ]}{[ {P}_{tot} ]} + {[ θ ]}_{mon} \frac{{n [ monomer ]}^{n}}{exp ( \frac{Δ G ( T )}{- RT} )} \frac{1}{[ {P}_{tot} ]} , \\ Δ G ( T ) = Δ {H}^{∘} - T Δ {S}^{∘} + {C}_{P} [ T - {T}^{∘} - T ln ( \frac{T}{{T}^{∘}} ) ]\end{matrix}$$

where [θ] is mean residue elipticity (deg cm² dmol⁻¹), T° is the midpoint of the transition, ΔH° is the van't Hoff enthalpy at the midpoint (kcal mol⁻¹), ΔS° is the standard state entropy (kcal mol⁻¹ K⁻¹), and ΔC_{P} is the change in heat capacity over the temperature range of the experiment (kcal mol⁻¹ K⁻¹). In fitting, the floating parameters are the initial and final [θ] values, the slopes of the folded and unfolded baselines, ΔH, ΔC_{P}, and Tₘ. The parameters were globally fit to three equilibria schemes, from monomer-tetramer to hexamer, using data collected at 49 and 126 μM. Only the monomer-pentamer scheme provided an adequate fit to the data. ΔC_{P} was held constant for pWSPLB at the value obtained for WSPLB.

Gel Filtration Chromatography

Analytical gel filtration chromatography was used to assess the distribution of oligomeric states in solution at various concentrations using a Superose G75 column (Amersham Biosciences). The dilution of the peak over the column was calculated to be 10-fold. All runs were performed in running buffer 25 mM sodium phosphate pH 7.0, 100 mM NaCl, 1 mM TCEP-HCl, and 1 mM EDTA using an FPLC (Amersham Biosciences). The column was calibrated using a 10 mg/ml solution of ovalbumin (43 kDa), chymotrypsin (25 kDa), cytochrome C (12.5 kDa) and aprotinin (6.5 kDa). WSPLB was loaded both at 458 (3.8 mg/ml) and 50 μM (0.31 mg/ml). WSPLB (residues 21-52) was run at 2.5 mM (10 mg/ml) and 250 μM (I mg/ml) both boiled and unboiled. Elution from the column was monitored at 280 nm wavelength with a UVM-II monitor (Amersham Biosciences).

Analytical Ultracentrifugation

Measurements were made at 25° C. using a Beckman XL-1 analytical ultracentrifuge. Samples of WSPLB (15, 39, and 113 μM in 100 mM NaCl, 25 mM MOPS pH 7.5, 1 mM EDTA and 1 mM TCEP-HCl) were centrifuged to equilibrium at 30, 35, and 40,000 rpm in six-channel, carbon-epoxy composite centerpieces supplied by Beckman. WSPLB (residues 21-52) samples (14, 46, and 97.5 μM in 15 mM MOPS pH 7.0, 50 mM NaCl, 1 mM TCEP-HCl, and 1 mM EDTA) were centrifuged at 48,000 rpm. Concentrations were monitored using absorption optics at a wavelength of 275 nm and equilibrium was assessed by the absence of significant change in radial concentration gradients in scans separated by a few hours. Peptide partial specific volumes, solvent densities, monomer molecular masses, and molar extinction coefficients were calculated using the program “SEDINTERP” (Laue, T., et al., Computer-aided interpretation of analytical sedimentation data for proteins, The Royal Society of Chemistry, Cambridge, U.K. (1992)) modified to use the amino acid partial specific volumes and molecular weights reported by Kharakoz (Kharakoz, D. P., Biochem. 36:10276-10285 (1997)). An uncertainty was estimated of about +10% in the calculated molecular weight of the protein, arising largely from uncertainty in the partial specific volume, which is calculated from a weight average of individual amino acids. Calculated values were held constant, and data at both initial concentrations and the different speeds were analyzed by global curve-fitting of error-weighted optical absorption data to the sedimentation equilibrium equation for monomer-nmer equilibrium. To obtain the oligomer size present, the molecular weight was fit to data from the most concentrated samples with a single molecular weight species fit using Igor-Pro® (WaveMetrics, Lake Oswego, Oreg., 97035) programs developed from a previous version (Brooks, I. S., et al., Biophys. J. 64:a244 (1993)). In these fits, baselines, signal values, and the molecular weight were allowed to vary. Association constants were determined similarly, keeping the monomer molecular weight and oligomer number constant, and allowing the equilibrium constant to float. After fitting, a species plot was calculated representing the contribution of each species to total signal as a function of concentration,

### II. Potassium Channel KcsA

Approximately 30% of the open reading frames of the genomes of higher eukaryotes code for proteins that span or are associated with cell membranes (Stevens, T. J. and Arkin, I. T., Proteins 39:417-420 (2000)). To date roughly 13,000 X-ray or NMR derived structures of water-soluble proteins have been deposited in the PDB, while only ˜35 structures of membrane proteins are currently known, due to inherent difficulties in membrane protein purification and crystallization. While membrane associated proteins are very difficult to characterize, water-soluble proteins are amenable to a wide range of biophysical experimental techniques. The interiors of integral membrane proteins and water-soluble proteins are similar (Rees, D. C., et al., Science 245:510-513 (1989)) in terms of amino acid composition and packing angles although some of the fine details differ (Bowie, J. U., J. Mol. Biol. 272:780-789 (1997); Eilers, M., et al., Biophys. J. 82:2702-2736 (2002)). The greatest difference between soluble and membrane-spanning proteins is the hydrophobicity of the amino acids on the exterior surface, where the amino acids that contact the lipid bilayer in membrane-spanning proteins are more hydrophobic relative to those seen on the surface of water-soluble proteins. Thus, a membrane-spanning protein can be made water-soluble by mutating its hydrophobic surface residues, if there is no alteration of the core. Such a technique allows one to bypass the membrane to study membrane protein structures, while addressing fundamental questions about the forces that stabilize the native states of both water and membrane-soluble proteins.

The three-dimensional structures of tens of thousands of water-soluble proteins have been solved, but the structures of membrane-soluble proteins have proven to be much more difficult to determine. The problems associated with membrane proteins include their limited levels of expression, low stability in detergent-solubilized forms, and a greater difficulty in obtaining high-resolution diffraction quality crystals. Thus, a broadly applicable automated strategy for the preparation of water-soluble mutants of membrane proteins is needed, which could be obtained in larger quantities for a variety of high-resolution biophysical and drug discovery studies. Initial work included a simple transmembrane coiled-coil (Li, H., et al., Biochemistry 40:6636-6645 (2001); Slovic, A. M., et al., Protein Science 12:337-348 (2002); Frank, S., et al., Biochemistry 39:6825-6831 (2000)), but attempts to design water-soluble versions of larger proteins such as bacteriorhodopsin led to misfolded proteins with very limited solubility in water (Mitra, K., et al., Protein Engineering 15:485-492 (2002)).

In another embodiment of the present invention, a computational approach is disclosed for the design of a water-solubilized version of a bacterial ion channel with a transplanted mammalian toxin-binding site. These findings have fundamental implications concerning the stabilization of membrane versus water-soluble proteins (Popot, J.-L. and Engelman, D., Biochemistry 29:4031-4037 (1990)) as well as practical implications for the design of water-soluble analogues of a variety of biologically interesting membrane proteins.

The structures of many membrane proteins are beginning to appear, allowing one to infer features that are common to entire families. In particular, the structures of bacterial ion channels (Doyle, D. A., et al., Science 280:69-77 (1998); Dutzler, R., et al., Nature 415:287-294 (2002); Bass, R. B., et al., Science 298:1582-1587 (2002); Chang, G., et al., Science 282:2220-2226 (1998); Jiang, Y., et al., Nature 417:515-522 (2002); Jiang, Y., et al., Nature 423:33-41 (2003); Zhou, Z., et al., Nature 414:43-48 (2001)) have provided insight into their mammalian counterparts, and the structure of rhodopsin (Palczewski, K., et al., Science 289:739-745 (2000)) has served as a prototype for the entire family of 7-transmembrane G-protein coupled receptors (GPCRs). It would be advantageous to use what structural information is available for a given membrane protein to obtain water-soluble versions that retain their structure, oligomerization state, and essential ligand-binding properties. The bacterial KcsA potassium channel was selected because of its available structure (Doyle, D. A., et al., Science 280:69-77 (1998); Zhou, Z., et al., Nature 414:43-48 (2001)), its biochemical characterization, and the interest in this family of channel proteins. Furthermore, the external vestibule of the ion-conducting pore of KcsA has been mutated to the corresponding residues in a mammalian channel (Q58A, T61S, R64D) to allow binding of agitoxin2 (AgTx₂), resulting in a protein (designated here as tKcsA) that binds AgTx₂ (MacKinnon, R., et al., Science 280:106-109 (1998)). This system extends multiple, clearly defined criteria for the successful design of a water-soluble version of this protein, which should: 1) be expressed at high level in a water-soluble form; 2) show the correct helical secondary structure; 3) associate to form tetramers; 4) bind AgTx₂ with high affinity, specificity, and in the appropriate stoichiometry; and 5) bind small molecule channel blockers such as tetraethylammonium chloride (TEA). The designed water-soluble variants of tKcsA are referred to as WSK-1, WSK-2, and WSK-3.

Potassium Channel KcsA Protein Design

A statistical, entropy-based formalism has been developed for identifying amino acid probabilities from a given backbone structure (Zou, J. and Saven, J. G., J. Mol. Biol. 296:281-294 (2000); Kono, H. and Saven, J. G., J. Mol. Biol.306:607-628 (2001)). This method takes as input a target structure, in this case a high resolution structure of KcsA (PDB identifier:1k4c), and energy functions that quantify sequence-structure compatibility. The output is the set of site-specific probabilities of the amino acids compatible with the structure. The site-specific probabilities of the amino acids and their discrete side chain conformational states (rotamers) are determined by maximizing an effective entropy function subject to simultaneous constraints on both the overall energy as determined by an atom-based potential and the value of an effective solvation score (“environmental energy”). In the calculation, all 20 amino acids and up to 10 of their side chain conformations (rotamer states (Dunbrack, R. L., Jr. and Cohen, F. E., Protein Science 6:1661-1681 (1997)) were considered at each site where mutations were permitted. This yields a total of 129 identity-rotamer states for each variable position. C₄ subunit symmetry was imposed, reducing the complexity (total number of possible sequence-rotamer combinations) from 129¹⁴⁰ to 129³⁵ for the first of the recursive calculations discussed below (Fu, X., et al., Protein Engineering (in press)).

To quantify hydrophobicity and solvation effects, an environmental potential is used (Kono, H. and Saven, J. G., J. Mol. Biol. 306:607-628 (2001)), where the local Cβ density about each residue is used to quanitify its degree of exposure to solvent. The environmental energy for the entire protein (Eₑₙᵥ) and for the buried residue sites (Eₑₙᵥ_{_{—}}_{b}) are constrained to the average values of water-soluble proteins in the calculation (−46.0 and −24.6). The identities of buried sites are constrained in these calculations, and the decrease in Eₑₙᵥ (from +20 to −46) largely results from the mutation of exposed hydrophobic residues to more hydrophilic amino acids.

Non-bonded interactions involving the side chains are calculated using the AMBER 3A force field (Weiner, S. J., et al., J. Am. Chem. Soc. 106:765-784 (1984)) with a modified hydrogen bonding term (Kono, H. and Doi, J., J. Computa. Chem. 17:1667-1683 (1996)) and a distance dependent dielectric constant (ε=4 r). To address unfolded states, a reference energy γ_{ref} (α) for each amino acid is introduced into the energy E_{c} to represent the effects of the denatured state. The energy is calculated as a “free energy” of each amino acid in its N-acetyl-N′-methylamide derivative with averaging over multiple backbone and rotamer states, This averaging involves a sum over possible rotamers and possible backbone configurations, approximated by varying each of the backbone φ and φ angles in increments of 10 degrees. This approximates an average over extended unfolded states. The reference energies of each amino acid may then be estimated using:

$$\begin{matrix}{γ}_{ref} ( α , {β}_{ref} ) = - {β}_{ref}^{- 1} ln ( {z}_{ref} ( α , {β}_{ref} ) / {z}_{ref} ( G , {β}_{ref} ) ) \\ {z}_{ref} ( α , {β}_{ref} ) = {\sum }_{φ , ϕ , k} exp ( - {β}_{ref} {ϵ}_{ref} ( φ , ϕ , {r}_{k} ( α ) ) )\end{matrix}$$

where γ_{ref} is the conformational energy in a particular conformation of the N-acetyl-N′-methylamide derivative of the amino acid as determined using the molecular potential. Here β_{ref}=1/k_{B}T, where k_{B} is Boltzmann's constant and T is a temperature appropriate for the conformation sampling of side chain and backbone conformations (e.g., T=300 K). Reference energies are expressed relative to Gly (Kono, H. and Saven, J. G., J. Mol. Biol. 306:607-628 (2001)). In the statistical formalism of scads, accompanying the inter-atomic potential energy is a corresponding effective temperature 1/β. The probabilities used in sequence identification were determined for β=0.5 mol/kcal; at this value we find the sequence properties are robust with respect to slight variation in backbone structure (Kono, H. and Saven, J. G., J. Mol. Biol. 306:607-628 (2001)).

WSK expression and purification

The gene encoding WSK-1 was synthesized with Pfu Polymerase using 3 fragments. The final WSK-1 fragment was cloned into the pET-24a(+) vector (Novagen, available from EMD Biosciences, Inc., Madison, Wis., 53719) at NdeI and XhoI restriction sites, expressing no tag. Mutants WSK-2 and WSK-3 were generated using QuikChange (Stratagene, La Jolla, Calif., 92037). The WSK proteins were expressed in BL21(DE3) cells (Novagen) in Luria Bertani (LB) broth for 4 hours after induction with 1 mM isopropyl-β-D-thiogalactoside. Cells were harvested by centrifugation at 4° C. and 5000 r.p.m. Cell pellets were lysed by French press at 1500 psi in lysis buffer containing 10 mM Tris[hydroxymethyl]aminomethane hydrochloride pH 7.0, 1 mM EDTA. Cell extracts were loaded onto a 40 ml Q Sepharose (Amersham Biosciences, Piscataway, N.J., 08855) column in the lysis buffer, and eluted with a step gradient of the lysis buffer containing 0-500 mM KCl. Fractions containing WSK were pooled and concentrated to 50 ml, and further purified by reverse-phase HPLC on a Vydac C4 preparative column using a linear gradient of water and acetonitrile containing 0.1% trifluoroacetic acid. Purity was verified by analytical reverse-phase HPLC, MALDI-TOF mass spectrometry, and 4-12% Bis-Tris SDS-PAGE gels. The molar extinction coefficient (ε_{280 nm}=17781 M⁻¹cm⁻¹) was determined by the difference in absorbance of a sample in 0 and 6 M Guanidine-HCl (GuHCl), using the calculated extinction coefficient at 280 nm as a starting point.

Agitoxin2 Synthesis and Purification

Agitoxin2 (AgTx₂, 4097 Da) was chemically synthesized as a C-terminal carboxyamide on a 0.25 mmol scale using an Applied Biosystems model 433A solid phase peptide synthesizer (Perkin-Elmer) with standard FMOC amino acid chemistry. The tripeptide gly-gly-N-2,4-dinitrophenyl-Ala chromophore was coupled to the N-terminus of half of the resin. AgTx₂-DNP has a calculated ε_{356 nm}=11,343 M⁻¹cm⁻¹, and ε_{280 nm}=4520 M⁻¹cm⁻¹. Peptides were washed on the resin with dimethylformamide and ether, cleaved for 1.5 hours with TFA:water:ethanedithiol:triisopropylsilane (94.5/2.5/2.5/1) v/v/v/v and precipitated and washed with cold ether. Purification proceeded by reverse-phase HPLC using a preparative C4 column (Vydac) and a linear gradient of the appropriate buffers. AgTx₂ required oxidative refolding to attain the proper folded state after HPLC. A series of redox buffers containing increasing proportions of oxidized (GSS) and reduced (GSSH) glutathione were made. The concentration of GSS was determined by UViis absorbance using ε_{282 nm}=273 M⁻¹cm⁻¹, while GSSH was monitored by titration with Elmans reagent, following absorbance at 412 nm. Optimal conditions were identical for both labeled and unlabeled peptides, and the final reaction components were determined to 125 μM toxin in the presence of air and 100% GSSH (6 mM). The progress of the reaction was followed using analytical reverse-phase HPLC and comparison of the magnitude of the properly folded peak to the misfolded peaks. Properly folded toxins were repurified by reverse-phase HPLC and tested for function.

Solution Measurements

CD spectra were collected on an AVIV 62DS spectropolarimeter, using a 1 mm pathlength quartz cuvette, with protein at 44 μM, in 20 mM potassium phosphate pH 7.0, 100 mM KCl, and 1 mM EDTA. For ultracentrifugation, samples were centrifuged at 15,000 r.p.m. in 20 mM potassium phosphate, pH 7.0, 100 mM KCl, and 1 mM EDTA using a Beckman XL-I analytical ultracentrifuge. Equilibrium dialysis to determine the stoichiometry of toxin binding to WSK-3 was performed using 0.5 ml centrifuge tubes, and a dialysis membrane with a 10 kDa molecular weight cutoff. After reaching equilibrium, the concentrations of AgTx2-DNP and WSK-3 were determined by absorbance at 356 nm and 280 nm respectively.

The extent of binding of AgTx₂-DNP (10 μM) to WSK-3 (10 μM, tetramer) was assessed at various concentrations of TEA and TMA by ultracentrifugation at 15,000 r.p.m. Because we expected some salt dependence to the binding interaction, we also measured the extent of binding in the presence of the same concentrations of KCl. The fraction of free AgTx₂-DNP was determined from the absorbance at 360 nm at the top of the cell (at this speed, the WSK-3 is essentially fully depleted, but the AgTx₂-DNP has not significantly sedimented). The extent of binding, relative to the KCl control, was plotted and the IC₅₀ determined (FIG. 5). The approximate K_{diss} for binding to TEA is determined from the relationship IC₅₀=K_{TEA} [f_{AgTx2-DNP}]/K_{AgTx2-DNP} where f_{AgTx2-DNP} is defined as the free AgTx₂-DNP concentration, and K_{TEA} and K_{AgTx2-DNP} are the dissociation constants for WSK-3 with TEA and AgTx₂-DNP respectively. This relationship holds approximately for [f_{AgTx2-DNP}] >>K_{AgTx2-DNP}, where [f_{AgTx2-DNP}]=5 μM and K_{AgTx2-DNP}≧2 μM (as tight binding is observed in the equilibrium dialysis with 10 μM total toxin concentration).

Design of Water Soluble Potassium Channel KcsA (WSK)

The side chains of the transmembrane helices of tKcsA were stripped to the backbone, and then rebuilt in accordance with the requirements for function, stability, and water-solubility. The pore-lining residues, the extracellular loops, the intracellular region, and the residues required for binding to AgTx₂ were retained. Furthermore, the buried residues were retained, leaving 35 membrane-exposed sidechains (greater than 40% accessible to a probe with a radius of 1.4 Å (Sridharan, S., et al., J. Computa. Chem. 16:1038-1044 (1995)) per protomer as targets for design. These positions were chosen using the computational design algorithm scads (statistical computationally assisted design strategy).

A key input into these calculations is an “environmental energy,” a database-derived quantification of solvation and hydrophobic effects (Kono, H. and Saven, J. G., J. Mol. Biol. 306:607-628 (2001)). The value of this environmental score for wild type KcsA is +20, which is well outside the range observed for soluble proteins of this size, due to the large number of exposed hydrophobic residues.

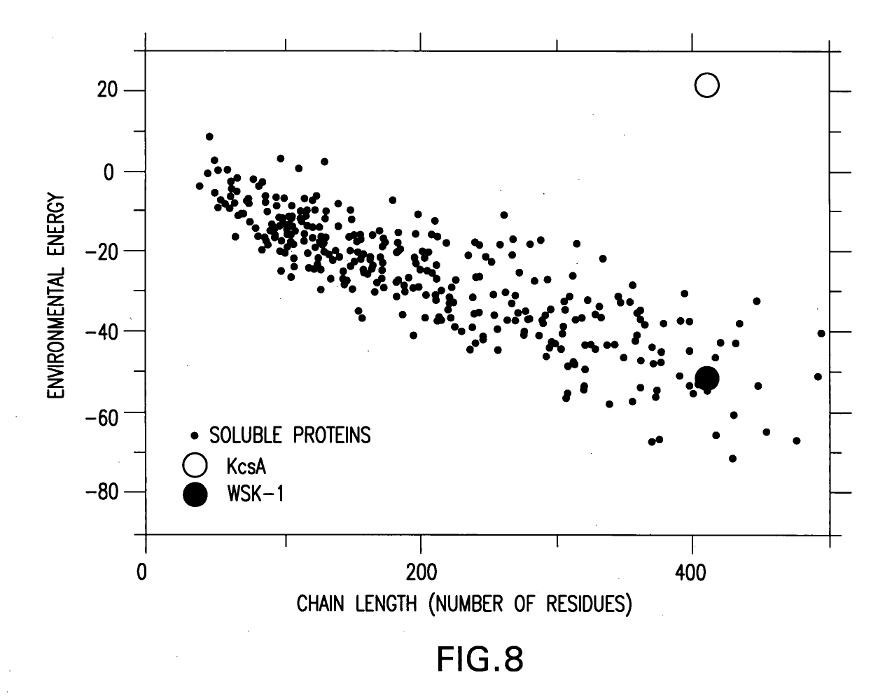

FIG. 8 shows environmental “energy” Eₑₙᵥ (Kono, H. and Saven, J. G., J. Mol. Biol. 306:607-628 (2001)) vs. chain length. Eₑₙᵥ quantifies solvation and hydrophobic effects and has been parameterized using a database of 500 soluble proteins (small circles) (Kono, H. and Saven, J. G., J. Mol. Biol. 306:607-628 (2001)). For each protein structure:

$${E}_{env} = {\sum }_{i} ϵ ( {a}_{i} , {r}_{a , i} , {ρ}_{C β} ) ;$$

where for the i^{th} residue aᵢ, r_{a,i}, and ρ_{cβ} are the amino acid, side chain conformation, and local density of beta carbons. Also shown is the Eₑₙᵥ for wild type KcsA (open circle) and the value Eₑₙᵥ that was used as a constraint in the sequence calculations (black circle). Thus, as shown in FIG. 8, in calculations to determine water-soluble sequences, the environmental energy was constrained to the value expected for soluble proteins of this size, −46.

Scads generates profiles describing the site-dependent probabilities of the amino acids at each position allowed to mutate. A unique sequence was selected from the computed probabilities using recursive calculations. The set of 35 exposed residues was initially targeted for variation. The identities of the remaining residues were fixed at wild type and their side chain conformations were permitted to vary in order to accommodate mutations (Zou, J. and Saven, J. G., J. Mol. Biol. 296:281-294 (2000); Kono, H. and Saven, J. G., J. Mol. Biol. 306:607-628 (2001)). A sequence was selected comprising the most probable amino acids at each site with the exception of several sites where alternate amino acids with appreciable probability were selected: W26E and G43A were chosen for their favorable helical propensities (Mufioz, V. and Serrano, L., Nature 1:399-409 (1994); Mufioz, V. and Serrano, L., J. Mol. Biol. 245:275-296 (1995), and S44, S69 and V106 were kept at wild type. The wild type amino acid was selected at 8 of the 35 sites. A second calculation was then performed. In the presence of the resulting designed sequence from the first iteration, an additional 8 exposed hydrophobic sites were targeted for mutation. The wild type was most probable at L36, V39, L40, I60, and L105; hydrophobic residues were probable at F116 and the wild type was retained. The mutation V93E was most probable, and the L24D mutation was selected over the more probable W and F due to its greater polarity. At position 104, Gly and Ala had comparable probabilities, and Ala was chosen for to its aqueous helix propensity. The resulting sequence was compared to 47 aligned K⁺-channel sequences from 8 prokaryotic and eukaryotic organisms (BLAST®, NCBI database).

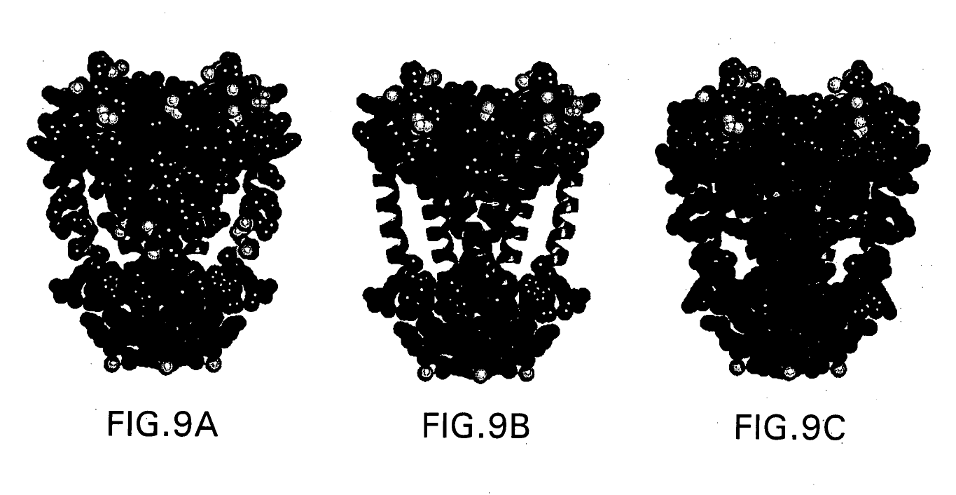

FIGS. 9A-C are a depiction of KcsA and WSK-3. Only the side chains of outer helices (22-71), inner helices (89-124), cytoplasmic, and extracellular residues are shown. Sidechains are colored based on their frequency of occurrence in the apolar section of the lipid bilayer beginning with most probable: Ala/Ile/Leu/Val (dark green), Gly/Met/Thr (light green), Pro/Ser/Trp/Tyr (light blue). Lys/Arg/Gln (dark blue), Asp/Glu (red). FIG. 9A is a depiction of KcsA. Lipid-exposed residues of KcsA allowed to vary in the design are depicted along the inner and outer helices (light green, dark green). Also shown are the cytoplasmic and extracellular residues, unchanged in our design. FIG. 9B shows the KcsA structure with sidechains of mutated residues removed. Extracellular and cytoplasmic residues that were held constant from KcsA to WSK are rendered. Buried residues within the interior of the structure are not shown. FIG. 9C shows the WSK-3. Sequences of KcsA and WSK-3 are also shown, where colored residues were mutated, while those in black were not. Mutations to tKcsA are shown in a grey box. Depictions shown in FIGS. 9A-C were made using PyMol (DeLano Scientific, San Carlos, Calif.).

The resulting sequence (WSK-1) had 29 mutations relative to tKcsA. Preliminary studies discussed below indicated that this soluble protein bound toxin, but formed high-order oligomers. The model of WSK-1 showed two exposed hydrophobic patches in the redesigned transmembrane helices, which might mediate aggregation. This problem was addressed in a new round of computational design varying only these exposed hydrophobic regions, resulting in WSK-2, shown in FIG. 9B, which contained the additional mutation L116R, and WSK-3, shown in FIG. 9C, which contained both L81R and L116R.

The WSK sequences result from the simultaneous imposition of effective energetic constraints on the solvation properties and the inter-atomic interactions of the flexible amino acid side chains. One might expect that simply mutating exposed hydrophobic residues to polar or charged amino acids might be sufficient to confer solubilization, but such approaches often yield misfolded proteins (Mitra, K., et al., Protein Engineering 15:485-492 (2002)). The calculations provide sequences with sterically-consistent, nontrivial patterning of amino acid identities. Among the polar mutations that were introduced, complementary charge interactions on the protein surface are apparent in FIG. 9A-C. Although the overall goal is to produce a water-soluble structure, it must also retain sufficient hydrophobic interactions to drive protein folding in an aqueous environment. Thus, some exposed hydrophobes are retained, and one mutation actually yields increased hydrophobic character (S102V). Others have noted the importance of careful consideration of interactions between surface residues in protein design (Marshall, S. A., et al., J. Mol. Biol. 316:189-199 (2002)).

Secondary and Quaternary Structure of WSK variants

WSK variants were expressed in high yield (20 mg/ml) and in soluble form in E. Coli. The circular dichroism (CD) spectra of the purified proteins were consistent with the expected secondary structure, showing minima at 208 and 222 nm ([θ₂₂₂]=−14,600 deg cm² dmol⁻¹). The computed α-helical content was about 50% (Chakrabartty, A., et al., Nature 351:586-588 (1991)), in good agreement with the helical content of 60% observed for KcsA (1k4c).

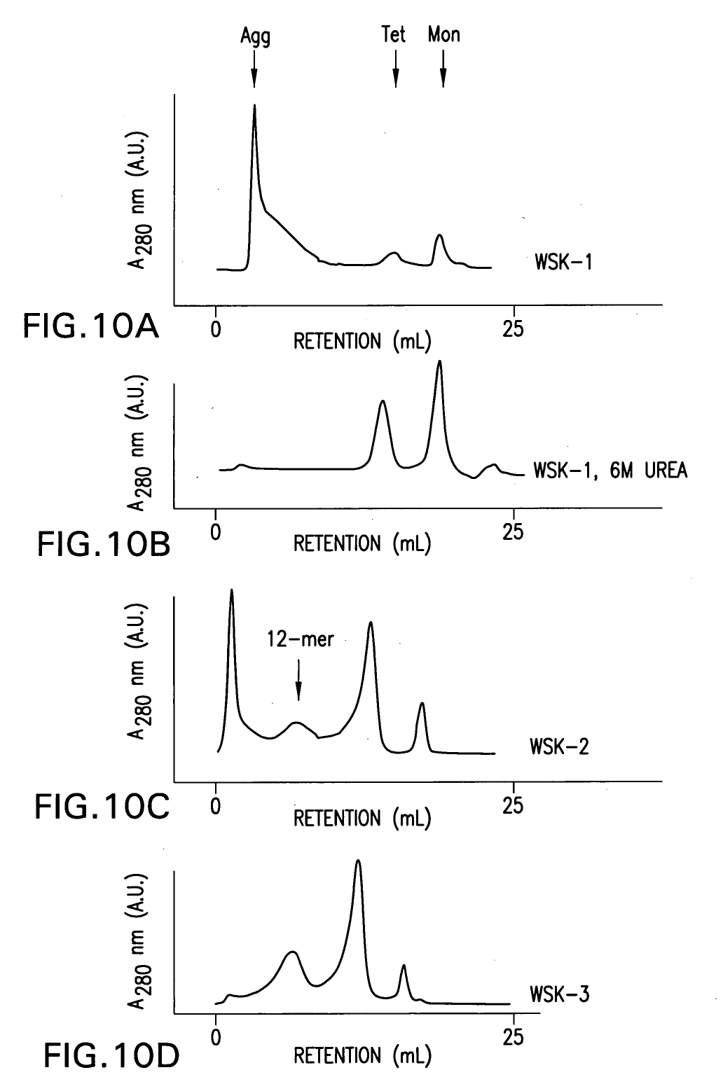

As shown in FIGS. 10A-D, size exclusion chromatography was used to determine the aggregation state of WSK variants. FIG. 10A shows a trace of WSK-1 (20 μM), FIG. 10B shows WSK-1 (20μM, 6 M Urea), FIG. 10C shows WSK-2 (100 μM), and FIG. 10D shows WSK-3 (100 μM). All traces were obtained using a 25 ml Superdex 200 column (Amersham Biosciences) in 20 mM K₂PO₄ pH 7.0, 100 mM KCl, and 1 mM EDTA at 1 ml/min flowrate. The approximate volumes of elution for the monomer (mon), tetramer (tet), 12-mer, and high-order aggregate (agg) are indicated. The column was calibrated using blue dextran, bovine serum albumin (66 kDa), ovalbumin (43 kDa), and carbonic anhydrase (29 kDa).

WSK-1 eluted as three peaks: one peak consistently eluted as a large aggregate in the void volume, while the observed molecular weights of the other two peaks were 10,600 Da and 50,100 Da, as shown in FIG. 10B, in good agreement with the expected masses for the monomer (11,433 Da) and tetramer (45,732 Da), respectively. Preliminary experiments (not shown) showed that both the tetramer as well as the higher order aggregate bound AgTx₂ in the proper stoichiometry, suggesting that the void volume peak might consist of loosely associated but otherwise properly folded tetramers. To test this possibility, the molecular weight distribution was measured in the presence of 6 M urea, a concentration lower than that required to unfold the protein (as determined from the loss of secondary structure followed by CD spectroscopy). Under this condition, only the tetrameric and monomeric peaks were observed, as shown in FIG. 10B.

WSK-1, WSK-2 and WSK-3 contain progressively fewer apolar sidechains in the re-designed transmembrane helices; these mutants also show a progressively smaller fraction of aggregated protein eluting in the void volume. Even in the absence of urea, WSK-3 elutes primarily as a tetramer and shows only a small peak near the position expected for a 12-mer, presumably a trimer of tetramers, as shown in FIG. 10C-D. Thus, iterative mutagenesis guided by computation and experiment was able to minimize the nonspecific aggregates of tetramers seen in WSK-1.

AgTx₂ Binding

Size exclusion chromatography was used to demonstrate that WSK-3 specifically bound to AgTx₂. For these experiments, we synthesized a variant of AgTx₂ with 2,4-dinitrophenyl-Ala (AgTx₂-DNP) at its N-terminus, allowing the toxin to be detected from its absorbance at 356 nm (ε₃₅₆ₘₘ=11,340 M⁻¹cm⁻¹). When AgTx₂-DNP was chromatographed through the Superdex 200 column, it eluted as a monomer, but when incubated with WSK variants (10 μM, assuming a functional tetramer) it co-eluted with the tetrameric and higher order aggregate peaks of WSK-1, WSK-2, and WSK-3 (data not shown) in approximately the expected molar ratio of one AgTx₂-DNP for every four molecules of WSK.

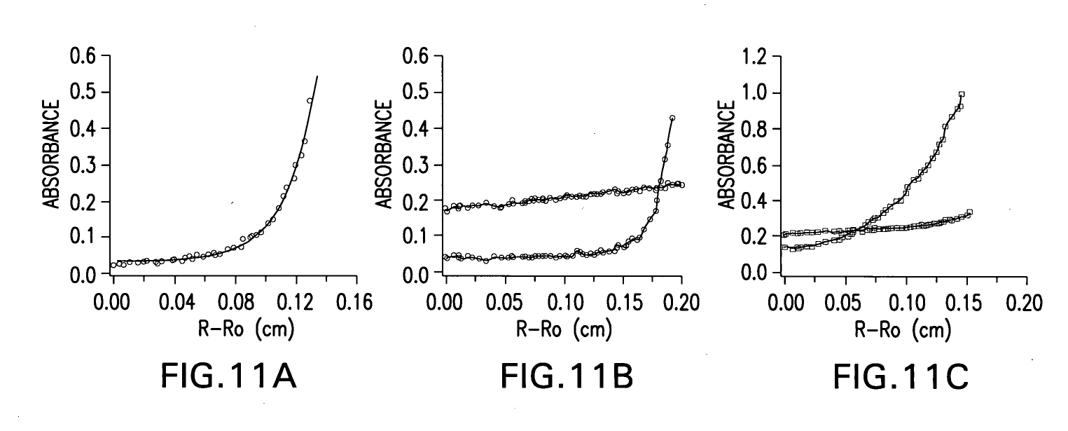

FIGS. 11A-C illustrate equilibrium sedimentation analytical ultracentrifugation of WSK-3 and AgTx₂-DNP. In FIG. 11A, 17 μM WSK-3 was monitored at 280 nm (o). Data were fit to a monomer-tetramer-12-mer equilibrium using a macro in Igor-Pro®. FIG. 11B shows 17 μM AgTx₂-DNP in the presence (o) and absence (o) of 17 μM WSK-3 tetramer, monitored at 360 nm to detect only the AgTx₂-DNP. FIG. 11C shows the control with BSA (36 μM) plus AgTx₂-DNP (17 μM) monitored at 280 nm (□) to monitor the BSA, and at 360 nm (□) to monitor AgTx₂-DNP.

Equilibrium analytical ultracentrifugation confirmed the selective association of WSK-3 with AgTx₂-DNP. WSK-3 sedimented in a concentration-dependent manner, and the traces are well defined by a monomer-tetramer-12-mer equilibrium (FIG. 11A), as expected from size exclusion chromatography. Under the same conditions, AgTx₂-DNP shows little sedimentation, consistent with its monomeric molecular weight (4,435 Da). When incubated with WSK-3, AgTx₂-DNP co-sediments with the water-solubilized channel (FIG. 11B) as determined by the DNP absorbance. At a mole ratio of one AgTx₂-DNP per WSK-3 tetramer (17 μM), there was essentially no free AgTx₂-DNP (Arkin, M. and Lear, J. D., Anal. Biochem. 299:98-107 (2001)), indicating that the dissociation constant for binding was <2 μM (i.e., 10-fold tighter than the total toxin concentration). By contrast, when AgTx₂-DNP was centrifuged in the presence of bovine serum albumin (BSA, 36 μM), no association was observed (FIG. 11C), demonstrating the specificity of the AgTx₂:WSK-3 interaction.

The stoichiometry of toxin binding was determined by equilibrium dialysis using two starting conditions: one with equimolar AgTx₂-DNP and WSK-3 tetramer (10 μM), as well as one with a 2-fold excess of AgTx₂-DNP. In both experiments the ratio of AgTx₂-DNP:WSK-3 monomer was 1:4 within experimental error (1:4.4 and 1:4.1 respectively). Control experiments showed no binding of AgTx₂-DNP to either BSA or carbonic anhydrase, shown in Table 2. In Table 2, WSK-3 and AgTx₂-DNP (Tox) initially are on side a, while buffer is on side b. At equilibrium, the concentration of bound toxin is given by [Tox]_{bound}=[Tox]ₐ-[Tox_{free}]ₐ=[Tox]ₐ-[Tox]_{b}.

TABLE 2
Equilibrium dialysis of WSK-3 and AgTx₂-DNP.

| Experiment | [Tox] bound | Stoichiometry |
|---|---|---|
| a) WSK-3ₘₒₙ (40 μM) + | 9.0 μM | [WSK-3]/[Tox]_{bound} = |
| Tox (10 μM) | | 4.4 |
| b) WSK-3ₘₒₙ (40 μM) + | 9.7 μM | [WSK-3]/[Tox]_{bound} = |
| Tox (20 μM) | | 4.1 |
| c) BSA (20 μM) + | 0 μM | |
| Tox (44 μM) | | |
| d) CA (47 μM) + | 0 μM | |
| Tox (23 μM) | | |

TEA competition

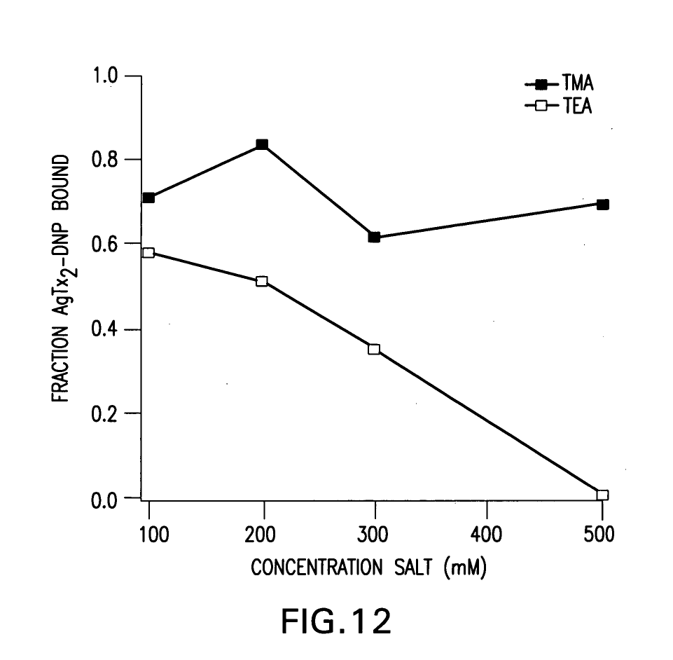

To determine whether small molecule channel blockers also bind specifically to WSK-3, the ability of TEA to compete with AgTx₂-DNP for binding to WSK-3 was measured. This small molecule blocker has been shown to compete with charybdotoxin (a homologue of AgTx₂) for binding to the extracellular opening of the pore (Miller, C., Neuron 1: 1003-1006 (1988)). TEA induces dissociation of AgTx₂-DNP from WSK-3 in a concentration-dependent manner, while the control tetramethylammonium ion (TMA, which does not bind to K⁺-channels (Heginbotham, L. and MacKinnon, R., Neuron 8:483-491 (1992))) showed no significant concentration-dependent competition, as shown in FIG. 12. From the concentration of TEA required to induce 50% total toxin dissociation, a K_{diss}≦80 mM was calculated for TEA (assuming a K_{diss}≦2 μM for binding of AgTx₂-DNP). This value agrees well with the reported K_{diss} of 3.2-22 mM (Heginbotham, L., et al., J. Gen. Phys. 114:551-559 (1999)) for the native channel. TEA binds to the outer vestibule of the channel KcsA channel. The finding that TEA binds to WSK-3 strongly suggests that this region of the protein is properly folded. Although this finding alone does not preclude the possibility of misfolding elsewhere in the protein, the additional CD, size exclusion chromatography, and toxin-binding data together suggest that the protein has a structure similar to the wild type channel.

Potassium Channel KscA Summary

WSK-3 has successfully recapitulated the target properties of tKcsA, including its ability to bind a protein toxin and a small molecule blocker. These findings should be of practical utility for the design of water-soluble analogues of membrane proteins that have proven to be difficult to obtain in large quantities for biophysical studies or drug screening. The method requires only a medium-resolution structure for computational modeling. Thus, it should be possible to extend this work to other ion channels and GPCR proteins (Becker, O. M., et al., Curr. Opin. Drug Discov. Devel. 6:353-361 (2003)) using homology models as starting points.

### III. Pharmaceutical Screening Methods

In another embodiment, the invention relates to a method of producing water soluble transmembrane proteins for pharmaceutical screening methods using the in-silico designed water-soluble transmembrane proteins. The water soluble proteins can be prepared using any method known to one skilled in the relevant art. For example, the protein can be synthesized chemically using a solid phase peptide synthesizer. In another example, the protein can be synthesized using recombinant techniques. The recombinant techniques include synthesizing a gene encoding for the in-silico designed water soluble transmembrane protein, cloning the gene and introducing the gene into a host cell. The protein can be synthesized in the host cell and isolated from the cell, or the protein is secreted from the host cell and then purified.

The water soluble proteins are isolated from the host cells in substantially purified form. Sufficient quantities of the purified proteins are produced to allow for its use in further studies. The purified water soluble transmembrane proteins can be used for a variety of pharmaceutical purposes, including, but not limited to, crystallization and other structural characterization methods; rational drug design, in-vitro drug screening, use of the protein as an antigen for antibody production and therapeutic applications including the development of vaccines.

In a specific example of structural characterization methods, diffraction crystals of the transmembrane portion of the water-solubilized phospholamban analogue can be obtained. Other structure determination methods include, but are not limited to multiwavelength anomalous diffraction (MAD), Single Isomorphous Replacement (SIR), Multiple Isomorphous Replacement (MIR), Single Isomorphous Replacement with Anomalous Scattering (SIRAS), Nuclear Magnetic Resonance (NMR) and other techniques.

The structural data determined using these methods can be used for further studies. For example, drug screening methods can be performed using the structural information. The methods include determining the transmembrane protein active binding site from the structural characterization, and designing biologically active agents that bind the active site. The biological agents can be any agents, including, but not limited to small organic and inorganic molecules, polymers, proteins, antibodies and other agents.

The produced water soluble proteins can be used in drug screening assays for screening for active agents. The active agents can induce or prevent a function of the water soluble protein. The water soluble protein relates to the native transmembrane protein and it is expected that agents that bind the water soluble analogue would also bind the native analogue. Therefore, the water soluble proteins can be used for drug screening methods for the design and discovery of novel biologically active agents that induce, inhibit or prevent functions of the native transmembrane protein. Such screening can employ the water soluble protein, nucleotides that encode the water soluble protein, nucleotides which hybridize to the nucleotides which encode water soluble protein, and combinations thereof.

The drug screening method includes a method of identifying potentially therapeutic compounds or agents comprising: (a) contacting a water soluble protein with one or more test compounds or agents; and (b) monitoring whether the one or more test compounds binds to the water soluble protein; wherein compounds or agents which bind the water soluble protein are potentially therapeutic compounds or agents.

The drug screening method relates to the use of partially or fully purified water soluble proteins which may be used in homogenous or heterogeneous binding assays to screen a large number or library of compounds and compositions for their potential ability to induce, inhibit or prevent one or more functions of the water soluble protein. And those compositions capable of binding to the water soluble protein are potentially useful for inducing, inhibiting or preventing one or more functions of the native transmembrane protein in vivo.

The drug screening method which is used in determining whether the compound or agent binds specifically to the water soluble protein, may comprise a competitive or noncompetitive homogeneous assay. The homogeneous assay may be a fluorescence polarization assay or a radioassay. Alternatively, determining whether the compound or agent binds specifically to the water soluble protein may comprise a competitive heterogeneous assay. The heterogeneous assay may be a fluorescence assay, a radioassay or an assay comprising avidin and biotin. The water soluble protein may comprise a detectable label. The label on the water soluble protein may be selected from the group consisting of a fluorescent label and a radiolabel. Alternatively, the compound or agent may comprise a detectable label. The label on the compound or agent may be selected from the group consisting of a fluorescent label and a radiolabel.

In another suitable assay, surface plasmon resonance is used to determine the binding of a molecule to the mutated or water-soluble protein. In this method, the water soluble protein is immobilized (usually by chemical reaction) on a stationary surface in a detection cell. The molecule to be analyzed is then passed over the stationary surface, and changes in the refractive index of the surface are monitored. A binding event is observed as an increase in the refractive index of the surface in proportion to the molecular mass of the molecule that binds to the surface. A suitable surface plasmon resonance system is a Biacore® system.

In one embodiment, the computer representation of the water soluble protein is used to discover a compound that binds to the mutated protein. The field of computational drug design provides tools and information. For example, see Krumrine, J., et al., “Principles and methods of docking and ligand design,” Methods Biochem. Anal. 44:443-76 (2003); Lyne, P. D., “Structure-based virtual screening: an overview,” Drug Discovery Today 7(20):1047-55 (2002); Xu H., “Retrospect and prospect of virtual screening in drug discovery,” Current Topics Medicinal Chemistry 2(12):1305-20 (2002); and Waszkowycz, B. “Structure-based approaches to drug design and virtual screening,” Curr. Opin. Drug. Discov. Devel. 5(3):407-13 (2002). By using the computer representation of the mutated, or water soluble, protein, one can design or identify a molecule that effectively inhibits, activates, or modulates the water soluble protein. The process may comprise one or more of the following: de novo design of a compound; structure-based design of a compound; molecular docking; and in silico library screening. A number of commercially available software programs are available for use in the present invention. See, for example, Dock™ (Ewing et al., J. Comput. Aided Mol. Des. 15:411-28 (2001)); AutoDock™(Scripps Research Institute; Morris, G. M., et al., J. Comp. Chem. 19: 1639-1662 (1998)); FlexX™ (Tripos, Inc.); FlexE (Claussen, H., et al., J. Mol. Biol.308:377-95 (2001); ICM™ (Internal Coordinate Mechanics); QXP™ (ThistleSoft); FLOG™; GOLD™; LUDI™ (Accelrys); X-Ligand™ (Accelrys, Inc.); and Glide (Schrodinger, Inc.).

The invention also relates to the use of the water soluble proteins for raising antibodies to the protein. Any method known to one skilled in the relevant art can be used to raise such antibodies. The antibodies can be used in a variety of pharmaceutical screening methods or in the production of vaccines. An alternative vaccine for use in the present invention comprises the water solubilized protein or a portion thereof, used as an antigen, to mount an immune response. The vaccines are used to inhibit or prevent the onset of an ailment or condition related to one or more functions of the native transmembrane proteins.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A computer based method for in-silico water-solubilization of a protein that normally resides in a membrane, comprising:
(1) mutating one or more aspects of a computer readable representation of the protein to confer water solubility to the protein while retaining a function of the protein; and
(2) outputting a computer representation of the mutated protein.

2. The method according to claim 1, wherein step (1) comprises:
(a) determining apolar regions of the membrane;
(b) determining residues of the protein that are normally in contact with the apolar regions of the membrane; and
(c) mutating at least one residue of the protein that is normally in contact with the apolar regions of the membrane to confer water solubility to the protein while retaining a folded structure of the protein.

3. The method according to claim 1, wherein step (1) comprises mutating a set of one or more side chains of the protein while retaining a biological function of the one or more side chains.

4. The method according to claim 1, wherein step (1) comprises:
(a) determining residues that are exposed on a transmembrane surface of the protein; and
(b) replacing one or more of the residues that are exposed on the transmembrane surface of the protein with one or more residues that confer water solubility on the protein.

5. The method according to claim 2, wherein step (1) comprises searching for combinations of amino acid side chains that provide water solubility and conformational stability to maintain a three dimensional structure of the protein.

6. The method according to claim 5, wherein step (1) further comprises:
(a) assigning side chains to one or more of the residues of the protein that are normally in contact with the apolar regions of the membrane;
(b) repeating step 6(a) for additional side chains;
(c) evaluating energies for combinations of the side chains assigned to the residues; and
(d) determining a set of one or more assigned side chains that provide a relatively low energy level.

7. The method according to claim 6, wherein assigned side chains are selected from a set of naturally occurring residues and placed in a low-energy conformation or rotamers.

8. The method according to claim 6, wherein the energies are evaluated for pair-wise combinations of assigned side chains using a potential function.

9. The method according to claim 8, wherein the potential function considers a net charge of amino acid side chains and a distance between C-beta atoms of the amino acid side chains.

10. The method according to claim 8, wherein the potential function considers a side chain-to-side chain interaction pairwise potential function.

11. The method according to claim 8, wherein the potential function considers one or more of the following:
van der Waals potential;
electrostatic interactions;
hydrogen bonding;
torsional energy;
bond angles; and/or
bond lengths.

12. The method according to claim 6, wherein the potential function considers environmental factors.

13. The method according to claim 12, wherein the environmental factors include solvation and/or hydrophobic effects.

14. The method according to claim 5, wherein step (1) comprises:
(a) selecting side chains to assign to the one or more residues of the protein that are normally in contact with the apolar regions of the membrane according to an evaluation of energy levels of the side chains that is performed using a simplified residue-based pair-wise potential evaluation; and
(b) assigning the selected side chains to the one or more of the residues of the protein that are normally in contact with the apolar regions of the membrane.

15. The method according to claim 14, wherein the simplified residue-based pair-wise potential evaluation comprises scoring energies based on a net charge of amino acid side chains and a distance between C-beta atoms of the amino acid side chains.

16. The method according to claim 15, wherein the energies are scored using a side chain-to-side chain interaction pairwise potential function.

17. The method according to claim 2, further comprising defining a set of amino acids that will water-solubilize the protein while maintaining a three-dimensional structure of the protein.

18. The method according to claim 17, further comprising searching possible combinations of side chains for low energy groupings.

19. The method according to claim 17, further comprising applying a search algorithm.

20. The method according to claim 17, wherein the search algorithm comprises a stochastic search algorithm

21. The method according to claim 17, wherein the stochastic search algorithm comprises one or more of the following:
Monte Carlo algorithm; and
genetic algorithm.

22. The method according to claim 17, wherein the search algorithm comprises a deterministic search algorithm.

23. The method according to claim 17, wherein the deterministic search algorithm comprises one or more of:
dead end elimination; and
branch and bound.

24. The method according to claim 17, wherein the search algorithm comprises a combination of stochastic and deterministic search algorithms.

25. The method according to claim 1, wherein the protein normally resides in a phospholipid membrane.

26. The method of claim 1, wherein the protein comprises a binding site for at least one biologically active agent.

27. The method of claim 26, wherein said mutated protein retains said binding site.

28. The method of claim 27, wherein said mutated protein retains the function of binding said at least one biologically active agent.

29. A computer based method for in-silico water-solubilization of a protein that normally resides in a membrane, comprising:
(1) providing a computer readable representation of the protein;
(2) determining residues of the transmembrane sequence of the protein;
(3) determining the lipid-exposed residues of the transmembrane sequence from the representation;
(3) selecting one or more or the lipid-exposed residues for mutation;
(4) mutating the one or more lipid-exposed residues with hydrophilic residues to form a mutated sequence;
(5) calculating a residue-based energy of the mutated sequence using a function that comprises one or more of the following terms:
intrinsic helical propensities of the amino acids;
intrahelical pairwise residue interaction energies;
interaction energy between the residues and helix macrodipole;
interhelical electrostatic interaction energy;
sidechain polarity;
a solubility term;
a method to compute van der Waals interactions and clashes;
a method to compute bond angles, lengths and torsional angles;
a method to compute hydrogen bonds; and
sequence entropy term;
(6) repeating steps (4) and (5) one or more times to minimize the residue-based energy function and form an optimized mutated sequence; and
(7) outputting a computer readable representation of the mutated protein.

30. The method of claim 29, wherein the method to compute van der Waals interactions and clashes comprises hard sphere approximations having 6-12 potentials.

31. The method of claim 29, further comprising after step (6):
removing steric clashes between residue side chains in the optimized mutated sequence.

32. The method of claim 29, wherein the protein is phospholamban.

33. A computer based method for in-silico water-solubilization of a protein that normally resides in a membrane, comprising:
(1) providing a computer readable representation of the protein;
(2) determining residues of the transmembrane sequence of the protein;
(3) determining the lipid-exposed residues of the transmembrane sequence from the representation;
(4) selecting one or more or the lipid-exposed residues for mutation using a SCADS algorithm;
(5) removing side chains from the selected residues of the transmembrane sequence;
(6) replacing the removed side chains with hydrophilic side chains;
(7) calculating the energy of the energy of the mutated sequence using a function comprising:
an environmental term; and
an interatomic amino acid side chain interaction term;
(8) repeating steps (4) and (5) one or more times to minimize the residue-based energy function and form an optimized mutated sequence; and
(9) outputting a computer readable representation of the mutated protein.

34. The method of claim 33, wherein the protein is potassium channel KcsA.

35. A computer program product comprising a computer usable medium having computer readable program code means embodied in said medium for causing an application program to execute on a computer that in-silico water-solubilizes a protein that normally resides in a membrane, said computer readable program code means comprising:
a first computer readable program code that causes the computer to mutate one or more aspects of a computer readable representation of the protein to confer water solubility to the protein while retaining a function of the protein; and
a second computer readable program code that causes the computer to output a computer representation of the mutated protein.

36. The computer program product of claim 35, further comprising:
a third computer readable program code that causes the computer to determine apolar regions of the membrane;
a fourth computer readable program code that causes the computer to determine residues of the protein that are normally in contact with the apolar regions of the membrane; and
a fifth computer readable program code that causes the computer to mutate at least one reside of the protein that is normally in contact with the apolar regions of the membrane to confer water solubility to the protein while retaining a native function of the protein.

37. The method of claim 1, further comprising after (2):
preparing the mutated protein.

38. The method of claim 37, wherein said preparing comprises:
chemically synthesizing the protein.

39. The method of claim 37, wherein said preparing comprises:
(a) synthesizing a gene for the mutated protein;
(b) cloning the gene;
(c) introducing the gene into a host cell; and
(d) expressing a water soluble protein from the gene in the host cell.

40. The method of claim 37, further comprising:
(f) crystallizing the water soluble protein; and
(g) determining the crystal structure of the protein.

41. The method of claim 40, further comprising:
(h) determining the structure of the active site of the protein; and
(i) designing a biologically active agent for binding the active site.

42. The method of claim 37, further comprising:
(j) screening a library of compounds or biologically active agents for a compound or agent that binds the active site of the protein.

43. The method of claim 37, further comprising:
raising antibodies to the water soluble protein.

44. The method of claim 43, further comprising:
producing a vaccine comprising the raised antibodies.

45. The method of claim 37, further comprising:
producing a vaccine comprising the water soluble protein or a portion thereof.

46. A method for producing a water soluble protein, the method comprising:
(1) mutating one or more aspects of a computer readable representation of the protein in-silico to confer water solubility to the protein while retaining a function of the protein;
(2) outputting a computer representation of the mutated protein; and
(3) preparing the mutated protein.

47. A method of identifying potentially therapeutic compounds or agents comprising:
(1) mutating one or more aspects of a computer readable representation of the protein in-silico to confer water solubility to the protein while retaining a function of the protein;
(2) outputting a computer representation of the mutated protein.
(3) preparing the mutated protein.
(4) contacting the water soluble protein with one or more test compounds or agents; and
(5) monitoring whether said one or more test compounds or agents binds to the water soluble protein;
wherein compounds which bind the water soluble protein are potentially therapeutic compounds or agents.

48. The method of claim 47, wherein (5) further comprises monitoring the binding using a competitive or noncompetitive homogeneous assay.

49. The method of claim 48, wherein said homogeneous assay is a fluorescence polarization assay, radioassay, or a surface plasmon resonance assay.

50. The method of claim 47, wherein (3) comprises:
preparing the protein using recombinant techniques.

51. The method of claim 1, further comprising in-silico screening of a library of compounds or biologically active agents for a compound or agent that binds the active site of the computer representation of the mutated protein.

52. A water soluble protein prepared according to the method of claim 1.

53. A water soluble analogue of the protein Phospholamban.

54. The water soluble analogue of claim 53, having essentially the same oligomerization states as native Phospholamban.

55. The water soluble analogue of claim 53, having the same response to phosphorylation as native Phospholamban.

56. A water soluble analogue of the potassium channel KcsA.
